(19) United States
(12) Patent Application Publication
(10) Pub. No.: US 2005/0085683 A1
(43) Pub. Date: Apr. 21, 2005

(54) Implantable heart assist system and method of applying same

(76) Inventors: Steven F. Bolling, Ann Arbor, MI (US); Kenneth Charhut, Lake Forest, CA (US); Anthony Viole, Foothill Ranch, CA (US); Marvin A. Konstam, Wayland, MA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/686,040

(22) Filed: Oct. 15, 2003

(51) Int. Cl. A61N 1/362

(52) U.S. Cl. 600/16

(57) ABSTRACT

An intravascular extracardiac pumping system for increasing perfusion through a renal artery to tissues of a patient without any component thereof being connected to the patient's heart is provided. The system includes means for pumping blood and a portion that houses the pumping means. The portion that houses the pumping means is configured to direct blood from a location upstream of the pumping means to a location within a renal artery. The pumping means and the portion that houses the pumping means are configured to be insertable into a non-primary vessel subcutaneously in an minimally-invasive procedure for positioning within the patient's vasculature.

### BACKGROUND OF THE INVENTION

1. Field of the Invention

This application relates generally to a system for assisting the heart and, in particular, to an extracardiac pumping system and a method for supplementing the circulation of blood through the patient, for enhancing vascular blood mixing, and for increasing perfusion to organs through branch arteries using a minimally invasive procedure.

2. Description of the Related Art

During the last decade, congestive heart failure (CHF) has burgeoned into the most important public health problem in cardiovascular medicine. As reported in Gilum, R. F., Epidemiology of Heart Failure in the U.S., 126 Am. Heart J. 1042 (1993), four hundred thousand (400,000) new cases of CHF are diagnosed in the United States annually. The disorder is said to affect nearly 5 million people in this country and close to 20 million people worldwide. The number of hospitalizations for CHF has increased more than three fold in the last 15 years. Unfortunately, nearly 250,000 patients die of heart failure annually. According to the Framingham Heart Study, the 5-year mortality rate for patients with congestive heart failure was 75 per cent in men and 62 per cent in women (Ho, K. K. L., Anderson, K. M., Kannel, W. B., et al., Survival After the Onset of Congestive Heart Failure in Framingham Heart Study Subject, 88 Circulation 107 (1993)). This disorder represents the most common discharge diagnosis for patients over 65 years of age. Although the incidence of most cardiovascular disorders has decreased over the past 10 to 20 years, the incidence and prevalence of congestive heart failure has increased at a dramatic rate. This number will increase as patients who would normally die of an acute myocardial infarction (heart attack) survive, and as the population ages.

CHF manifests itself primarily by exertional dyspnea (difficult or labored breathing) and fatigue. Three paradigms are used to describe the causes and therapy of CHF. The first views this condition in terms of altered pump function and abnormal circulatory dynamics. Other models describe it largely in terms of altered myocardial cellular performance or of altered gene expression in the cells of the atrophied heart. In its broadest sense, CHF can be defined as the inability of the heart to pump blood throughout the body at the rate needed to maintain adequate blood flow, and many of the normal functions of the body.

To address CHF, various cardiac assist devices have been developed. A cardiac or circulatory assist device is one that aids the failing heart by increasing its pumping function or by allowing it a certain amount of rest to recover its pumping function. Because congestive heart failure may be chronic or acute, different categories of heart assist devices exist. One type of chronic heart assist system employs a full or partial prosthetic connected between the heart and the aorta, one example of which is commonly referred to as a Left Ventricular Assist Device (LVAD). The LVAD is intended to take over for the left ventricle, and thus must pump blood at cardiac rates. With an LVAD, oxygenated blood circulation is established sufficient to satisfy the demand of the patient's organs.

Typically, the pump associated with older LVADs was a bulky pulsatile flow pump, of the pusher plate or diaphragm style, such as those manufactured by Baxter Novacor or TCI, respectively. Given that the pump was implanted within the chest and/or abdominal cavity, major invasive surgery was required. Alternatively, rotary pumps, such as centrifugal or axial pumps, have been used in heart assist systems. With centrifugal pumps, the blood enters and exits the pump practically in the same plane. An axial pump, in contrast, directs the blood along the axis of rotation of the rotor. Inspired by the Archimedes screw, one design of an axial pump has been miniaturized to about the size of a pencil eraser, although other designs are larger. Despite its small size, an axial pump may be sufficiently powerful to produce flows that approach those used with older LVADs. Even with miniaturized pumps, however, the pump is typically introduced into the left ventricle through the aortic valve or through the apex of the heart, and its function must be controlled from a console outside the body through a driveline.

The above and other common heart assist systems have several general features in common: 1) the devices are cardiac in nature; i.e., they are placed directly within or adjacent to the heart, or within one of the primary vessels associated with the heart (aorta), and are often attached to the heart and/or aorta; 2) the devices generally attempt to reproduce pulsatile blood flow naturally found in the mammalian circulatory system and, therefore, require valves to prevent backflow; 3) the devices are driven from external consoles, often triggered by the electrocardiogram of the patient; and 4) the size of the blood pump, including its associated connectors and accessories, is generally unmanageable within the anatomy and physiology of the recipient. Due to having one or more of these features, the prior art heart assist devices are limited in their effectiveness and/or practicality.

### SUMMARY OF THE INVENTION

It would be advantageous, therefore, to employ a heart assist system that avoids major invasive surgery and also avoids the use of peripheral equipment that severely restricts a patient's movement. It would also be advantageous to have such a heart assist system that can be employed in a non-hospital setting for ease of treating acute heart problems under emergency conditions.

In one embodiment, an intravascular extracardiac pumping system for increasing perfusion of tissue of a patient through a renal artery is provided. The system includes a pump and a pump housing. The pump is configured to pump blood through the patient at subcardiac volumetric rates. The pump has an average flow rate that, during normal operation thereof, is substantially below that of the patient's heart when healthy. The pump is configured to be positioned within the vasculature of a patient. The pump housing includes an inflow portion and an outflow portion. The inflow portion defines an axis. The inflow portion is fluidly coupled to the pump to direct blood to the pump. The inflow portion is configured to be positioned within the vasculature of the patient. The outflow portion extends generally laterally from the axis of the inflow portion. The outflow portion is fluidly coupled to the pump to direct blood away from the pump. The outflow portion is configured to be at least partially positioned within the renal artery of the patient. The pump and the inflow and outflow portions are configured so as to be inserted subcutaneously into the vasculature in a minimally-invasive procedure.

In another embodiment, an intravascular extracardiac pumping system is provided for increasing perfusion through a renal artery to tissues of a patient without any component thereof being connected to the patient's heart. The system includes means for pumping blood and a portion that houses the pumping means. The portion that houses the pumping means is configured to direct blood from a location upstream of the pumping means to a first location within a first artery and to a second location within a renal artery. The pumping means and the portion that houses the pumping means are configured to be insertable into a non-primary vessel subcutaneously in an minimally-invasive procedure for positioning within the patient's vasculature.

In another embodiment, a method for treating a patient without connecting any component to the patient's heart is provided. An inlet end of an inflow portion of an intravascular pumping system is inserted into the vasculature of a patient using a minimally invasive surgical procedure. The intravascular pumping system comprises a pump coupled with the inflow portion and an outflow portion coupled with the pump. The intravascular pumping system is advanced within the vasculature until the inlet end of the inflow portion is positioned at a first location within an artery and an outlet end of the outflow portion is positioned within a renal artery. The pump is operated to pump blood through the renal artery to perfuse tissue at volumetric rates that are on average subcardiac.

### BRIEF DESCRIPTION OF THE DRAWINGS

These and other features and advantages of the invention will now be described with reference to the drawings, which are intended to illustrate and not to limit the invention.

FIG. 1 is a schematic view of one embodiment of a heart assist system having multiple conduits for multi-site application, shown applied to a patient's vascular system;

FIG. 2 is a schematic view of another application of the embodiment of FIG. 1;

FIG. 3 is a schematic view of another embodiment of a heart assist system having multiple conduits for multi-site application wherein each of the conduits is applied to more than one vessel, shown applied to a patient's vascular system;

FIG. 4 is a schematic view of another embodiment of a heart assist system having multiple conduits for multi-site application and employing a connector with a T-shaped fitting, shown applied to a patient's vascular system;

FIG. 5 is a schematic view of an L-shaped connector coupled with an inflow conduit, shown inserted within a blood vessel;

FIG. 6 is a schematic view of another embodiment of a heart assist system having multiple conduits for multi-site application, shown applied to a patient's vascular system;

FIG. 7 is a schematic view of another application of the embodiment of FIG. 6, shown applied to a patient's vascular system;

FIG. 8 is a schematic view of another application of the embodiment of FIG. 6, shown applied to a patient's vascular system;

FIG. 9 is a schematic view of another embodiment of a heart assist system having multiple conduits for multi-site application, a reservoir, and a portable housing for carrying a portion of the system directly on the patient;

FIG. 10 is a schematic view of another embodiment of a heart assist system having a multilumen cannula for single-site application, shown applied to a patient's vascular system;

FIG. 11 is a schematic view of a modified embodiment of the heart assist system of FIG. 10, shown applied to a patient's vascular system;

FIG. 12 is a schematic view of another embodiment of a heart assist system having multiple conduits for single-site application, shown applied to a patient's circulatory system;

FIG. 13 is a schematic view of another application of the embodiment of FIG. 12, shown applied to a patient's vascular system;

FIG. 14 is a schematic view of one application of an embodiment of a heart assist system having an intravascular pump enclosed in a protective housing, wherein the intravascular pump is inserted into the patient's vasculature through a non-primary vessel;

FIG. 15 is a schematic view of another embodiment of a heart assist system having an intravascular pump housed within a conduit having an inlet and an outlet, wherein the intravascular pump is inserted into the patient's vasculature through a non-primary vessel;

FIG. 16 is a schematic view of a modified embodiment of the heart assist system of FIG. 15 in which an additional conduit is shown adjacent the conduit housing the pump, and in which the pump comprises a shaft-mounted helical thread;

FIG. 17 is a schematic view of a modified embodiment of the heart assist system of FIG. 15 in which a pump housing has an outflow portion that may extend into a renal artery;

FIG. 18 is a schematic view of another modified embodiment of the heart assist system of FIG. 15 in which a pump housing has a first outflow conduit that may extend into the left renal artery and a second outflow conduit that may extend into the right renal artery;

FIG. 19 is a schematic view of another modified embodiment of the heart assist system of FIG. 15 in which a pump housing has a Y-shaped outflow portion; and

FIG. 20 is a schematic view of another modified embodiment of the heart assist system of FIG. 15 in which a pump housing has an outflow portion located at the distal end thereof.

### DETAILED DESCRIPTION OF THE PREFERRED EMBODIMENT

Turning now to the drawings provided herein, more detailed descriptions of various embodiments of heart assist systems and cannulae for use therewith are provided below.

### I. Extracardiac Heart Assist Systems and Methods

A variety of heart assist systems and methods for supplementing the circulation of blood through the patient, for enhancing vascular blood mixing, and for increasing perfusion to organs through branch arteries using minimally invasive procedures are disclosed and claimed herein. Such systems preferably are extracardiac in nature. In other words, the systems supplement blood perfusion, without the need to interface directly with the heart and, in some cases, the aorta. Thus, the systems can be applied without major invasive surgery. The systems also preferably lessen the hemodynamic burden or workload on the heart by reducing afterload, impedence, and/or left ventricular end diastolic pressure and volume (preload). The systems also advantageously increase peripheral organ perfusion and provides improvement in neurohormonal status.

As discussed more fully below, the systems can be applied using one or more cannulae, one or more vascular grafts, one ore more pump housings, and a combination of one or more cannulae, one or more vascular grafts, and one or more pump housings. The systems employing cannula(e) and/or pump housings can be applied through multiple percutaneous insertion sites (sometimes referred to herein as a multi-site application) or through a single percutaneous insertion site (sometimes referred to herein as a single-site application).

A. Heart Assist Systems and Methods Employing Multi-Site Application

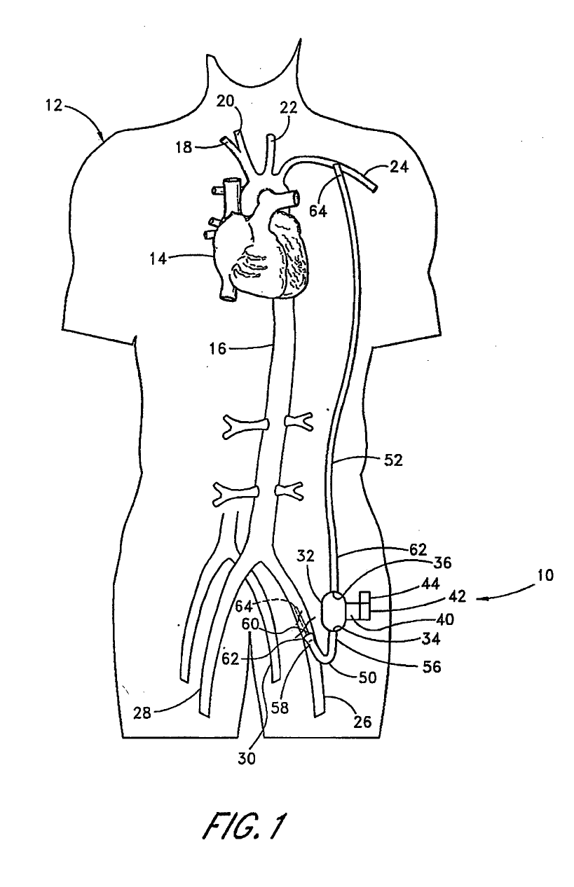

With reference to FIG. 1, a first embodiment of a heart assist system 10 is shown applied to a patient 12 having an ailing heart 14 and an aorta 16, from which peripheral brachiocephalic blood vessels extend, including the right subclavian artery 18, the right carotid artery 20, the left carotid artery 22, and the left axillary artery 24. Extending from the descending aorta is another set of peripheral blood vessels, the left and right iliac arteries which transition into the left and right femoral arteries 26, 28, respectively. As is known, each of the arteries 16, 18, 20, 22, 24, 26, and 28 generally conveys blood away from the heart. The vasculature includes a venous system that generally conveys blood to the heart. As will be discussed in more detail below, the heart assist systems described herein can also be applied to non-primary veins, including the left femoral vein 30.

The heart assist system 10 comprises a pump 32, having an inlet 34 and an outlet 36 for connection of conduits thereto. The pump 32 preferably is a rotary pump, either an axial type or a centrifugal type, although other types of pumps may be used, whether commercially-available or customized. The pump 32 preferably is sufficiently small to be implanted subcutaneously and preferably extrathoracically, for example in the groin area of the patient 12, without the need for major invasive surgery. Because the heart assist system 10 is an extracardiac system, no valves are necessary. Any inadvertent backflow through the pump 32 and/or through the inflow conduit would not harm the patient 12.

Regardless of the style or nature chosen, the pump 32 is sized to generate blood flow at subcardiac volumetric rates, less than about 50% of the flow rate of an average healthy heart, although flow rates above that may be effective. Thus, the pump 32 is sized and configured to discharge blood at volumetric flow rates anywhere in the range of 0.1 to 3 liters per minute, depending upon the application desired and/or the degree of need for heart assist. For example, for a patient experiencing advanced congestive heart failure, it may be preferable to employ a pump that has an average subcardiac rate of 2.5 to 3 liters per minute. In other patients, particularly those with minimal levels of heart failure, it may be preferable to employ a pump that has an average subcardiac rate of 0.5 liters per minute or less. In yet other patients it may be preferable to employ a pump that is a pressure wave generator that uses pressure to augment the flow of blood generated by the heart.

In one embodiment, the pump 32 is a continuous flow pump which superimposes a continuous blood-flow on the pulsatile aortic blood-flow. In another embodiment, the pump 32 has the capability of synchronous actuation; i.e., it may be actuated in a pulsatile mode, either in copulsating or counterpulsating fashion.

For copulsating action, it is contemplated that the pump 32 would be actuated to discharge blood generally during systole, beginning actuation, for example, during isovolumic contraction before the aortic valve opens or as the aortic valve opens. The pump 32 would be static while the aortic valve is closed following systole, ceasing actuation, for example, when the aortic valve closes.

For counterpulsating actuation, it is contemplated that the pump 32 would be actuated generally during diastole, ceasing actuation, for example, before or during isovolumic contraction. Such an application would permit and/or enhance coronary blood perfusion. In this application, it is contemplated that the pump 32 would be static during the balance of systole after the aortic valve is opened, to lessen the burden against which the heart must pump. The aortic valve being open encompasses the periods of opening and closing, wherein blood is flowing therethrough.

It should be recognized that the designations copulsating and counterpulsating are general identifiers and are not limited to specific points in the patient's heart cycle when the pump 32 begins and discontinues actuation. Rather, they are intended to generally refer to pump actuation in which the pump 32 is actuating, at least in part, during systole and diastole, respectively. For example, it is contemplated that the pump 32 might be activated to be out of phase from true copulsating or counterpulsating actuation described herein, and still be synchronous, depending upon the specific needs of the patient or the desired outcome. One might shift actuation of the pump 32 to begin prior to or after isovolumic contraction or to begin before or after isovolumic relaxation.

Furthermore, the pulsatile pump may be actuated to pulsate asynchronously with the patient's heart. Typically, where the patient's heart is beating irregularly, there may be a desire to pulsate the pump 32 asynchronously so that the perfusion of blood by the heart assist system 10 is more regular and, thus, more effective at oxygenating the organs. Where the patient's heart beats regularly, but weakly, synchronous pulsation of the pump 32 may be preferred.

The pump 32 is driven by a motor 40 and/or other type of drive means and is controlled preferably by a programmable controller 42 that is capable of actuating the pump 32 in pulsatile fashion, where desired, and also of controlling the speed or output of the pump 32. For synchronous control, the patient's heart would preferably be monitored with an EKG in which feedback would be provided the controller 42. The controller 42 is preferably programmed by the use of external means. This may be accomplished, for example, using RF telemetry circuits of the type commonly used within implantable pacemakers and defibrillators. The controller may also be autoregulating to permit automatic regulation of the speed, and/or regulation of the synchronous or asynchronous pulsation of the pump 32, based upon feedback from ambient sensors monitoring parameters, such as pressure or the patient's EKG. It is also contemplated that a reverse-direction pump be utilized, if desired, in which the controller is capable of reversing the direction of either the drive means or the impellers of the pump. Such a pump might be used where it is desirable to have the option of reversing the direction of circulation between two blood vessels.

Power to the motor 40 and the controller 42 may be provided by a power source 44, such as a battery, that is preferably rechargeable by an external induction source (not shown), such as an RF induction coil that may be electromagnetically coupled to the battery to induce a charge therein. Alternative power sources are also possible, including a device that draws energy directly from the patient's body; e. g., the patient's muscles, chemicals or heat. The pump can be temporarily stopped during recharging with no appreciable life threatening effect, because the system only supplements the heart, rather than substituting for the heart.

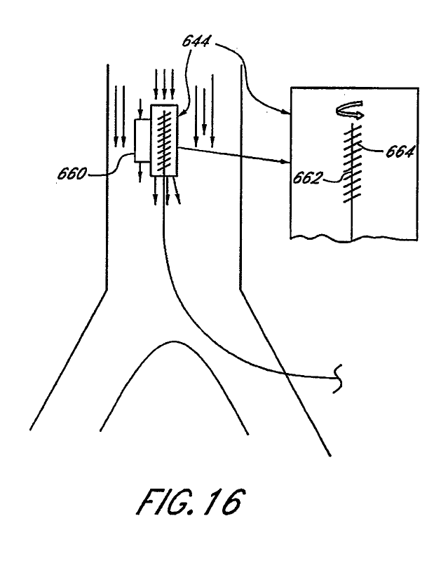

While the controller 42 and power source 44 are preferably pre-assembled to the pump 32 and implanted therewith, it is also contemplated that the pump 32 and motor 40 be implanted at one location and the controller 42 and the power source 44 be implanted in a separate location. In one alternative arrangement, the pump 32 may be driven externally through a percutaneous drive line or cable, as shown in FIG. 16. In another variation, the pump, motor and controller may be implanted and powered by an extracorporeal power source. In the latter case, the power source could be attached to the side of the patient to permit fully ambulatory movement.

The inlet 34 of the pump 32 is preferably connected to an inflow conduit 50 and an outflow conduit 52 to direct blood flow from one peripheral blood vessel to another. The conduits 50, 52 preferably are flexible conduits, as discussed more fully below. The conduits 50, 52 are coupled with the peripheral vessels in different ways in various embodiments of the heart assist system 10. As discussed more fully below, at least one of the conduits 50, 52 can be connected to a peripheral vessel, e.g., as a graft, using an anastomosis connection, and at least one of the conduits 50, 52 can be coupled with the same or another vessel via insertion of a cannula into the vasculature. Also, more than two conduits are used in some embodiments, as discussed below.

The inflow and outflow conduits 50, 52 may be formed from Dacron, Hemashield, Gortex, PVC, polyurethane, PTFE, ePTFE, nylon, or PEBAX materials, although other synthetic materials may be suitable. The inflow and outflow conduits 50, 52 may also comprise biologic materials or pseudobiological (hybrid) materials (e. g., biologic tissue supported on a synthetic scaffold). The inflow and outflow conduits 50, 52 are preferably configured to minimize kinks so blood flow is not meaningfully interrupted by normal movements of the patient or compressed easily from external forces. In some cases, the inflow and/or outflow conduits 50, 52 may come commercially already attached to the pump 32. Where it is desired to implant the pump 32 and the conduits 50, 52, it is preferable that the inner diameter of the conduits 50, 52 be less than 25 mm, although diameters slightly larger may be effective.

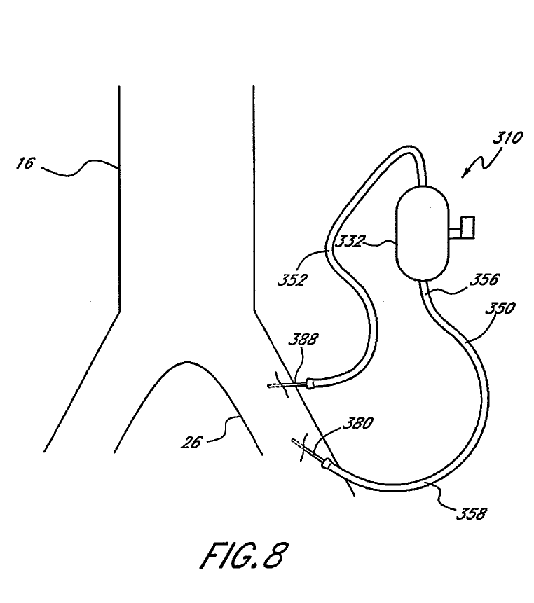

In one preferred application, the heart assist system 10 is applied in an arterial-arterial fashion; for example, as a femoral-axillary connection, as is shown in FIG. 1. It should be appreciated by one of ordinary skill in the art that an axillary-femoral connection would also be effective using the embodiments described herein. Indeed, it should be recognized by one of ordinary skill in the art that the present invention might be applied to any of the peripheral blood vessels in the patient. Another application of the heart assist system 10 couples the conduits 50, 52 with the same non-primary vessel in a manner similar to the application shown in FIG. 8 and discussed below.

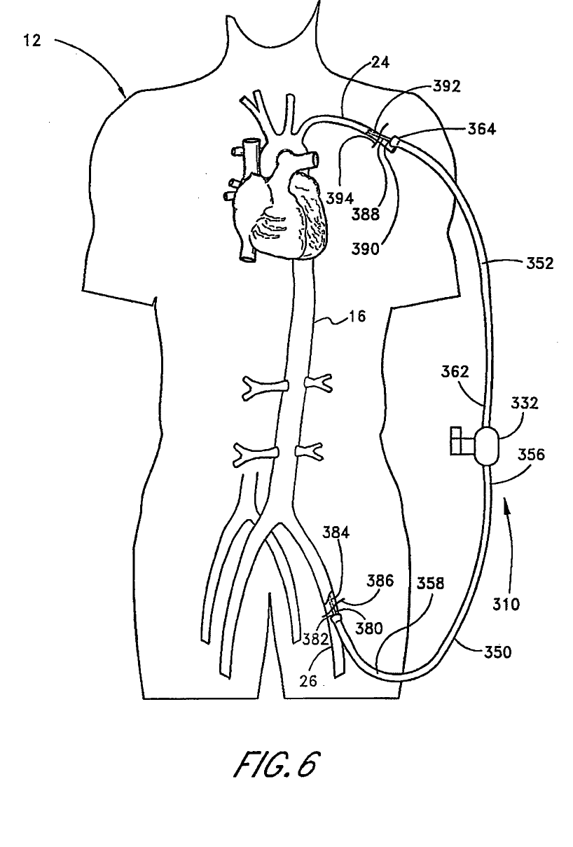

FIG. 1 shows that the inflow conduit 50 has a first end 56 that connects with the inlet 34 of the pump 32 and a second end 58 that is coupled with a, first non-primary blood vessel (e.g., the left femoral artery 26) by way of an inflow cannula 60. The inflow cannula 60 has a first end 62 and a second end 64. The first end 62 is sealably connected to the second end 58 of the inflow conduit 50. The second end 64 is inserted into the blood vessel (e.g., the left femoral artery 26). Although shown as discrete structures in FIG. 1, one skilled in the art would recognize that the inflow conduit 50 and the cannula 60 may be unitary in construction. While the cannula 60 preferably takes any suitable form, several modifications thereof may be found in U.S. patent application Ser. No. 10/078,283, filed Feb. 14, 2002, entitled A MULTILUMEN CATHETER FOR MINIMIZING LIMB ISCHEMIA, which is hereby expressly incorporated by reference in its entirety and made a part of this specification.

Where the conduit 50 is at least partially extracorporeal, the inflow cannula 60 also may be inserted through a surgical opening (e.g., as shown in FIG. 6 and described in connection therewith) or percutaneously, with or without an introducer sheath (not shown). In other applications, the inflow cannula 60 could be inserted into the right femoral artery or any other peripheral artery.

FIG. 1 shows that the outflow conduit 52 has a first end 66 that connects to the outlet 36 of the pump 32 and a second end 68 that connects with a second peripheral blood vessel, preferably the left axillary artery 24 of the patient 12, although the right axillary artery, or any other peripheral artery, would be acceptable. In one application, the connection between the outflow conduit 52 and the second blood vessel is via an end-to-side anastomosis, although a side-to-side anastomosis connection might be used mid-stream of the conduit where the outflow conduit were connected at its second end to yet another blood vessel or at another location on the same blood vessel (neither shown). Preferably, the outflow conduit 52 is attached to the second blood vessel at an angle that results in the predominant flow of blood out of the pump 32 proximally toward the aorta 16 and the heart 14, such as is shown in FIG. 1, while still maintaining sufficient flow distally toward the hand to prevent limb ischemia.

In another embodiment, the inflow conduit 50 is connected to the first blood vessel via an end-to-side anastomosis, rather than via the inflow cannula 60. The inflow conduit 50 could also be coupled with the first blood vessel via a side-to-side anastomosis connection mid-stream of the conduit where the inflow conduit were connected at its second end to an additional blood vessel or at another location on the same blood vessel (neither shown). Further details of these arrangements and other related applications are described in U.S. application Ser. No. 10/289,467, filed Nov. 6, 2002, the entire contents of which is hereby incorporated by reference in its entirety and made a part of this specification.

In another embodiment, the outflow conduit 52 also is coupled with the second blood vessel via a cannula, as shown in FIG. 6. This connection may be achieved in a manner similar to that shown in FIG. 1 in connection with the first blood vessel.

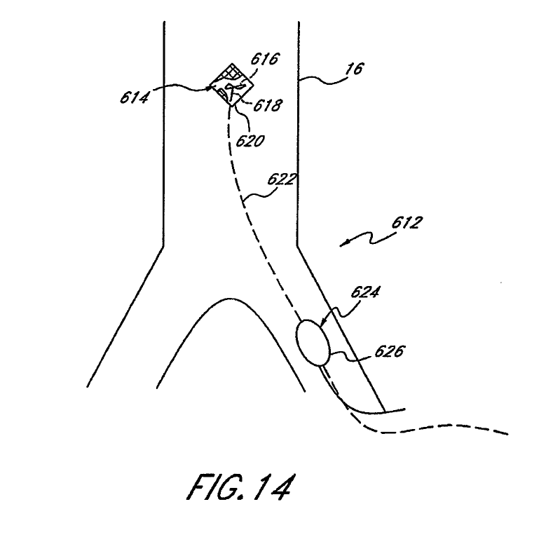

It is preferred that application of the heart assist system 10 to the peripheral or non-primary blood vessels be accomplished subcutaneously; e.g., at a shallow depth just below the skin or first muscle layer so as to avoid major invasive surgery. It is also preferred that the heart assist system 10 be applied extrathoracically to avoid the need to invade the patient's chest cavity. Where desired, the entire heart assist system 10 may be implanted within the patient 12, either extravascularly, e.g., as in FIG. 1, or at least partially intravascularly, e.g., as in FIGS. 14-20.

In the case of an extravascular application, the pump 32 may be implanted, for example, into the groin area, with the inflow conduit 50 fluidly connected subcutaneously to, for example, the femoral artery 26 proximate the pump 32. The outflow conduit would be tunneled subcutaneously through to, for example, the left axillary artery 24. In an alternative arrangement, the pump 32 and associated drive and controller could be temporarily fastened to the exterior skin of the patient, with the inflow and outflow conduits 50, 52 connected percutaneously. In either case, the patient may be ambulatory without restriction of tethered lines.

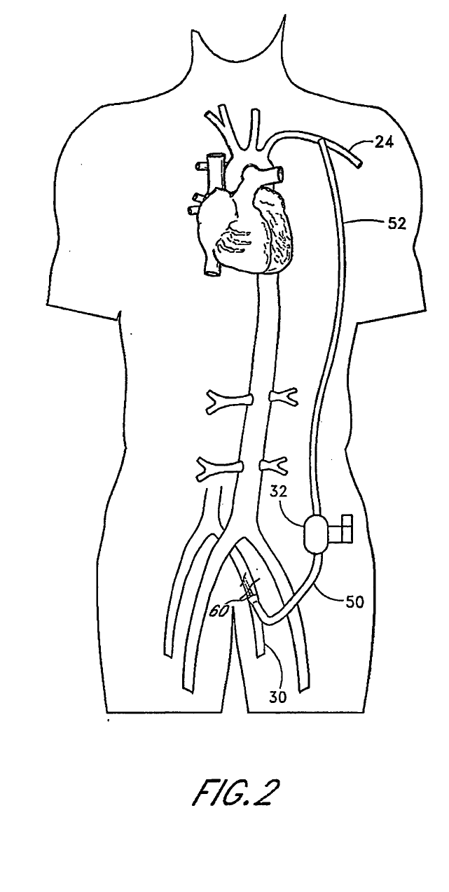

While the heart assist system 10 and other heart assist systems described herein may be applied to create an arterial-arterial flow path, given the nature of the heart assist systems, i. e., supplementation of circulation to meet organ demand, a venous-arterial flow path may also be used. For example, with reference to FIG. 2, one application of the heart assist system 10 couples the inflow conduit 50 with a non-primary vein of the patient 12, such as the left femoral vein 30. In this arrangement, the outflow conduit 50 may be fluidly coupled with one of the peripheral arteries, such as the left axillary artery 24. Arterial-venous arrangements are contemplated as well. In those venous-arterial cases where the inflow is connected to a vein and the outflow is connected to an artery, the pump 32 should be sized to permit flow sufficiently small so that oxygen-deficient blood does not rise to unacceptable levels in the arteries. It should be appreciated that the connections to the non-primary veins could be by one or more approach described above for connecting to a non-primary artery. It should also be appreciated that the present invention could be applied as a venous-venous flow path, wherein the inflow and outflow are connected to separate peripheral veins. In addition, an alternative embodiment comprises two discrete pumps and conduit arrangements, one being applied as a venous-venous flow path, and the other as an arterial-arterial flow path.

When venous blood is mixed with arterial blood either at the inlet of the pump or the outlet of the pump the ratio of venous blood to arterial blood should be controlled to maintain an arterial saturation of a minimum of 80% at the pump inlet or outlet. Arterial saturation can be measured and/or monitored by pulse oximetry, laser doppler, colorimetry or other methods used to monitor blood oxygen saturation. The venous blood flow into the system can then be controlled by regulating the amount of blood allowed to pass through the conduit from the venous-side connection.

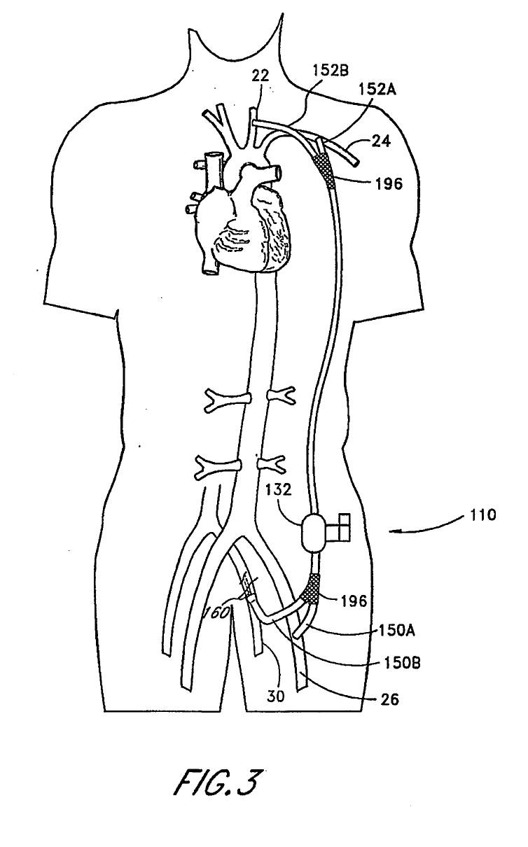

FIG. 3 shows another embodiment of a heart assist system 110 applied to the patient 12. For example, the heart assist system 110 includes a pump 132 in fluid communication with a plurality of inflow conduits 150A, 150B and a plurality of outflow conduits 152A, 152B. Each pair of conduits converges at a generally Y-shaped convergence 196 that converges the flow at the inflow end and diverges the flow at the outflow end. Each conduit may be connected to a separate peripheral blood vessel, although it is possible to have two connections to the same blood vessel at remote locations. In one arrangement, all four conduits are connected to peripheral arteries. In another arrangement, one or more of the conduits could be connected to veins. In the arrangement of FIG. 3, the inflow conduit 150A is connected to the left femoral artery 26 while the inflow conduit 150B is connected to the left femoral vein 30. The outflow conduit 152A is connected to the left axillary artery 24 while the outflow conduit 152B is connected to the left carotid artery 22. Preferably at least one of the conduits 150A, 150B, 152A, and 152B is coupled with a corresponding vessel via a cannula. In the illustrated embodiment, the inflow conduit 150B is coupled with the left femoral vein 30 via a cannula 160. The cannula 160 is coupled in a manner similar to that shown in FIG. 2 and described in connection with the cannula 60. The cannula 160 preferably takes any suitable form and modifications thereof are suggested by U.S. patent application Ser. No. 10/078,283, incorporated by reference hereinabove.

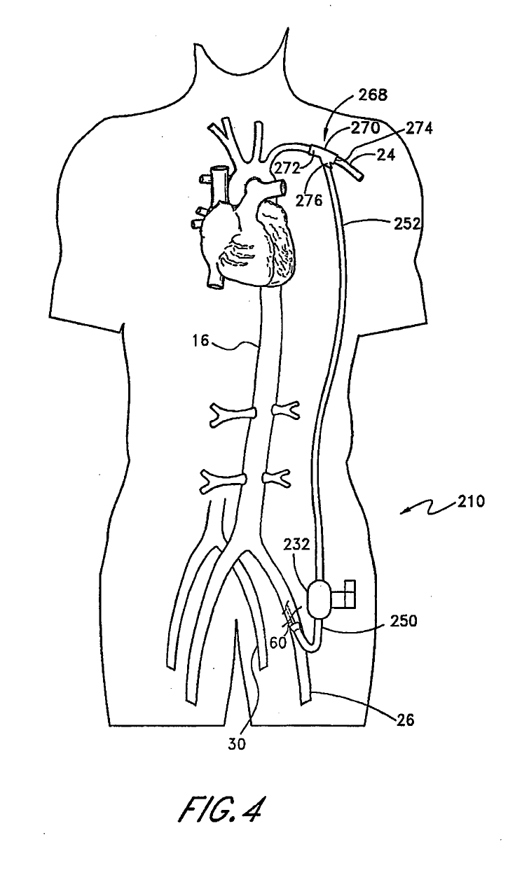

The connections of any or all of the conduits of the system 110 to the blood vessels may be via an anastomosis connection or via a connector, as described below in connection with FIG. 4. In addition, the embodiment of FIG. 3 may be applied to any combination of peripheral blood vessels that would best suit the patient's condition. For example, it may be desired to have one inflow conduit and two outflow conduits or vice versa. It should be noted that more than two conduits may be used on the inflow or outflow side, where the number of inflow conduits is not necessarily equal to the number of outflow conduits.

It is contemplated that, where an anastomosis connection is not desired, a connector may be used to connect at least one of the inflow conduit and the outflow conduit to a peripheral blood vessel. With reference to FIG. 4, an embodiment of a heart assist system 210 is shown, wherein an outflow conduit 252 is connected to a non-primary blood vessel, e.g., the left axillary artery 24, via a connector 268 that comprises a three-opening fitting. In one embodiment, the connector 268 comprises an intra-vascular, generally T-shaped fitting 270 having a proximal end 272 (with respect to the flow of blood in the left axillary artery and therethrough), a distal end 274, and an angled divergence 276 permitting connection to the outflow conduit 252 and the left axillary artery 24. The proximal and distal ends 274, 276 of the fittings 272 permit connection to the blood vessel into which the fitting is positioned, e.g., the left axillary artery 24. The angle of divergence 276 of the fittings 272 may be 90 degrees or less in either direction from the axis of flow through the blood vessel, as optimally selected to generate the needed flow distally toward the hand to prevent limb ischemia, and to insure sufficient flow and pressure toward the aorta to provide the circulatory assistance and workload reduction needed while minimizing or avoiding endothelial damage to the blood vessel. In another embodiment, the connector 268 is a sleeve (not shown) that surrounds and attaches to the outside of the non-primary blood vessel where, within the interior of the sleeve, a port to the blood vessel is provided to permit blood flow from the outflow conduit 252 when the conduit 252 is connected to the connector 268.

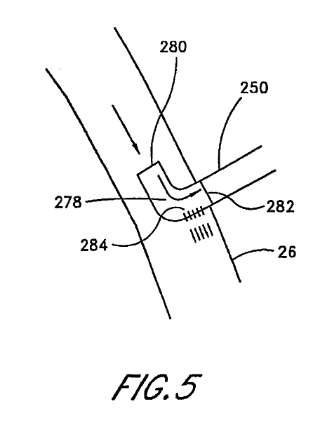

Other types of connectors having other configurations are contemplated that may avoid the need for an anastomosis connection or that permit connection of the conduit(s) to the blood vessel(s). For example, it is contemplated that an L-shaped connector be used if it is desired to withdraw blood more predominantly from one direction of a peripheral vessel or to direct blood more predominantly into a peripheral vessel. Referring to FIG. 5, the inflow conduit 250 is fluidly connected to a peripheral vessel, for example, the left femoral artery 26, using an L-shaped connector 278. Of course the system 210 could be configured so that the outflow conduit 252 is coupled to a non-primary vessel via the L-shaped connector 278 and the inflow conduit 250 is coupled via a cannula, as shown in FIG. 3. The L-shaped connector 278 has an inlet port 280 at a proximal end and an outlet port 282 through which blood flows into the inflow conduit 250. The L-shaped connector 278 also has an arrangement of holes 284 within a wall positioned at a distal end opposite the inlet port 280 so that some of the flow drawn into the L-shaped connector 278 is diverted through the holes 284, particularly downstream of the L-shaped connector 278, as in this application. A single hole 284 in the wall could also be effective, depending upon size and placement. The L-shaped connector 278 may be a deformable L-shaped catheter percutaneously applied to the blood vessel or, in an alternative embodiment, be connected directly to the walls of the blood vessel for more long term application. By directing some blood flow downstream of the L-shaped connector 278 during withdrawal of blood from the vessel, ischemic damage downstream from the connector may be avoided. Such ischemic damage might otherwise occur if the majority of the blood flowing into the L-shaped connector 278 were diverted from the blood vessel into the inflow conduit 252. It is also contemplated that a connection to the blood vessels might be made via a cannula, wherein the cannula is implanted, along with the inflow and outflow conduits.

One advantage of discrete connectors manifests in their application to patients with chronic CHF. A connector eliminates a need for an anastomosis connection between the conduits 250, 252 and the peripheral blood vessels where it is desired to remove and/or replace the system more than one time. The connectors could be applied to the first and second blood vessels semi-permanently, with an end cap applied to the divergence for later quick-connection of the present invention system to the patient. In this regard, a patient might experience the benefit of the heart assist systems described herein periodically, without having to reconnect and redisconnect the conduits 250, 252 from the blood vessels via an anastomosis procedure each time. Each time it is desired to implement any of the embodiments of the heart assist system, the end caps would be removed and a conduit attached to the connector(s) quickly.

In the preferred embodiment of the connector 268, the divergence 276 is oriented at an acute angle significantly less than 90 degrees from the axis of the T-shaped fitting 270, as shown in FIG. 4, so that a majority of the blood flowing through the outflow conduit 252 into the blood vessel (e. g., left axillary artery 24) flows in a direction proximally toward the heart 14, rather than in the distal direction. In an alternative embodiment, the proximal end 272 of the T, shaped fitting 270 may have a diameter larger than the diameter of the distal end 274, without need of having an angled divergence, to achieve the same result.

With or without a connector, with blood flow directed proximally toward the aorta 16, the result may be concurrent flow down the descending aorta, which will result in the reduction of afterload, impedence, and/or reducing left ventricular end diastolic pressure and volume (preload). Thus, the heart assist systems described herein may be applied so to reduce the afterload on the patient's heart, permitting at least partial if not complete CHF recovery, while supplementing blood circulation. Concurrent flow depends upon the phase of operation of the pulsatile pump and the choice of second blood vessel to which the outflow conduit is connected.

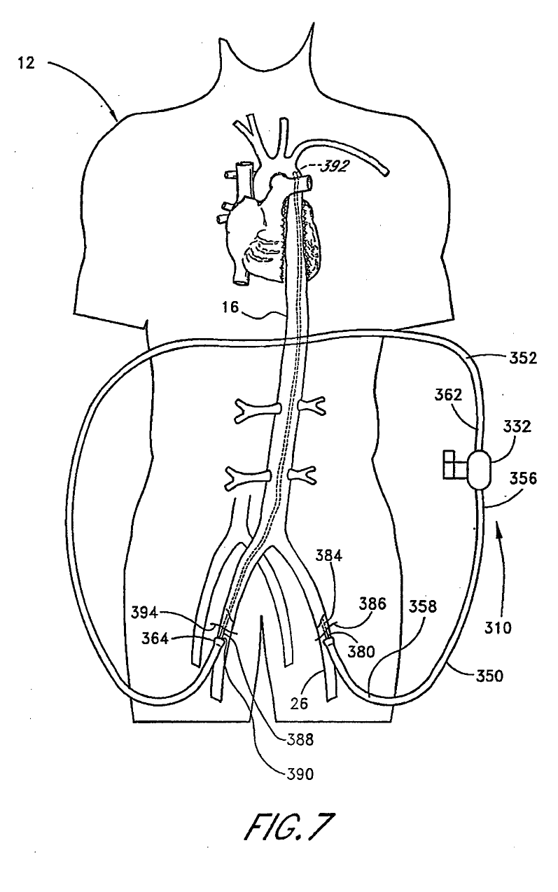

A partial external application of the heart assist systems is contemplated where a patient with heart failure is suffering an acute decompensation episode; i.e., is not expected to last long, or in the earlier stages of heart failure (where the patient is in New York Heart Association Classification (NYHAC) functional classes II or III). With reference to FIGS. 6 and 7, another embodiment of a heart assist system 310 is applied percutaneously to a patient 312 to connect two non-primary blood vessels wherein a pump 332 and its associated driving means and controls are employed extracorporeally. The pump 332 has an inflow conduit 350 and an outflow conduit 352 associated therewith for connection to two non-primary blood vessels. The inflow conduit 350 has a first end 356 and a second end 358 wherein the second end 358 is connected to a first non-primary blood vessel (e.g., femoral artery 26) by way of an inflow cannula 380. The inflow cannula 380 has a first end 382 sealably connected to the second end 358 of the inflow conduit 350. The inflow cannula 380 also has a second end 384 that is inserted through a surgical opening 386 or an introducer sheath (not shown) and into the blood vessel (e.g., the left femoral artery 26).

Similarly, the outflow conduit 352 has a first end 362 and a second end 364 wherein the second end 364 is connected to a second non-primary blood vessel (e.g., the left axillary artery 24, as shown in FIG. 6, or the right femoral artery 28, as shown in FIG. 7) by way of an outflow cannula 388. Like the inflow cannula 380, the outflow cannula 388 has a first end 390 sealably connected to the second end 364 of the outflow conduit 352. The outflow cannula 388 also has a second end 392 that is inserted through surgical opening 394 or an introducer sheath (not shown) and into the second blood vessel (e.g., the left axillary artery 24 or the right femoral artery 28). The cannulae 380 and 388 preferably take any suitable form. Several particularly useful modifications thereof are suggested in U.S. patent application Ser. No. 10/078,283, incorporated by reference hereinabove.

As shown in FIG. 7, the second end 392 of the outflow cannula 388 may extend well into the aorta 16 of the patient 12, for example, proximal to the left subclavian artery. If desired, it may also terminate within the left subclavian artery or the left axillary artery, or in other blood vessels, such as the mesenteric or renal arteries (not shown), where in either case, the outflow cannula 388 has passed through at least a portion of a primary artery (in this case, the aorta 16). Also, if desired, blood drawn into the extracardiac system 310 described herein may originate from the descending aorta (or an artery branching therefrom) and be directed into a blood vessel that is neither the aorta nor pulmonary artery. By use of a percutaneous application, the heart assist system 310 may be applied temporarily without the need to implant any aspect thereof or to make anastomosis connections to the blood vessels.

An alternative variation of the embodiment of FIG. 6 may be used where it is desired to treat a patient periodically, but for short periods of time each occasion and without the use of special connectors. With this variation, it is contemplated that the second ends of the inflow and outflow conduits 350, 352 be more permanently connected to the associated blood vessels via, for example, an anastomosis connection, wherein a portion of each conduit proximate to the blood vessel connection is implanted percutaneously with a removable cap enclosing the externally-exposed first end (or an intervening end thereof) of the conduit external to the patient. When it is desired to provide a circulatory flow path to supplement blood flow, the removable cap on each exposed percutaneously-positioned conduit could be removed and the pump (or the pump with a length of inflow and/or outflow conduit attached thereto) inserted between the exposed percutaneous conduits. In this regard, a patient may experience the benefit of the present invention periodically, without having to reconnect and redisconnect the conduits from the blood vessels each time.

Specific methods of applying this alternative embodiment may further comprise coupling the inflow conduit 352 upstream of the outflow conduit 350 (as shown in FIG. 8), although the reverse arrangement is also contemplated. It is also contemplated that either the cannula 380 coupled with the inflow conduit 350 or the cannula 388 coupled with the outflow conduit 352 may extend through the non-primary blood vessel to a second blood vessel (e.g., through the left femoral artery 26 to the aorta 16 proximate the renal branch) so that blood may be directed from the non-primary blood vessel to the second blood or vice versa.

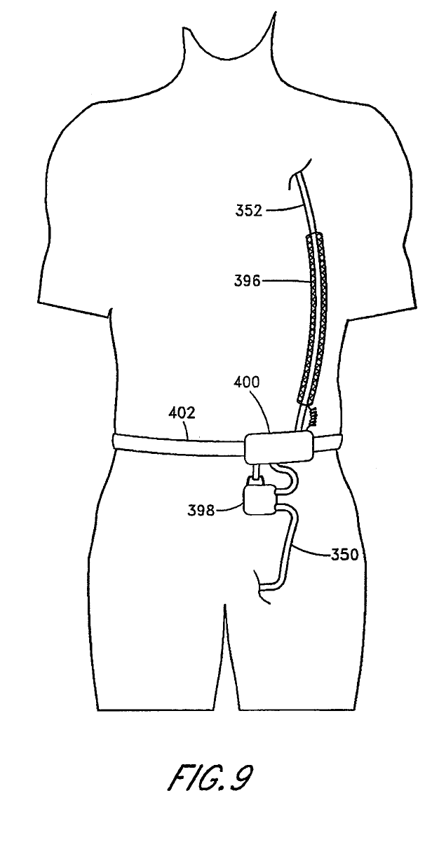

It is contemplated that a means for minimizing the loss of thermal energy in the patient's blood be provided where any of the heart assist systems described herein are applied extracorporeally. Such means for minimizing the loss of thermal energy may comprise, for example, a heated bath through which the inflow and outflow conduits pass or, alternatively, thermal elements secured to the exterior of the inflow and outflow conduits. Referring to FIG. 9, one embodiment comprises an insulating wrap 396 surrounding the outflow conduit 352 having one or more thermal elements passing therethrough. The elements may be powered, for example, by a battery (not shown). One advantage of thermal elements is that the patient may be ambulatory, if desired. Other means that are known by persons of ordinary skill in the art for ensuring that the temperature of the patient's blood remains at acceptable levels while travelling extracorporeally are also contemplated.

If desired, the present inventive system may further comprise a reservoir that is either contained within or in fluid communication with the inflow conduit. This reservoir is preferably made of materials that are nonthrombogenic. Referring to FIG. 9, a reservoir 398 is positioned fluidly in line with the inflow conduit 350. The reservoir 398 serves to sustain adequate blood in the system when the pump demand exceeds momentarily the volume of blood available in the peripheral blood vessel in which the inflow conduit resides until the pump output can be adjusted. The reservoir 398 reduces the risk of excessive drainage of blood from the peripheral blood vessel, which may occur when cardiac output falls farther than the already diminished baseline level of cardiac output, or when there is systemic vasodilation, as can occur, for example, with septic shock. It is contemplated that the reservoir 398 would be primed with an acceptable solution, such as saline, when the present system is first applied to the patient.

As explained above, one of the advantages of several embodiments of the heart assist system is that such systems permit the patient to be ambulatory. If desired, the systems may be designed portably so that it may be carried directly on the patient. Referring to FIG. 9, this may be accomplished through the use of a portable case 400 with a belt strap 402 to house the pump, power supply and/or the controller, along with certain portions of the inflow and/or outflow conduits, if necessary. It may also be accomplished with a shoulder strap or other techniques, such as a backpack or a fanny pack, that permit effective portability. As shown in FIG. 9, blood is drawn through the inflow conduit 350 into a pump contained within the portable case 400, where it is discharged into the outflow conduit 352 back into the patient.

B. Heart Assist Systems and Methods Employing Single-Site Application

As discussed above, heart assist systems can be applied to a patient through a single cannulation site. Such single-site systems can be configured with a pump located outside the vasculature of a patient, e.g., as extravascular pumping systems, inside the vasculature of the patient, e.g., as intravascular systems, or a hybrid thereof, e.g., partially inside and partially outside the vasculature of the patient.

1. Single-Site Application of Extravascular Pumping Systems

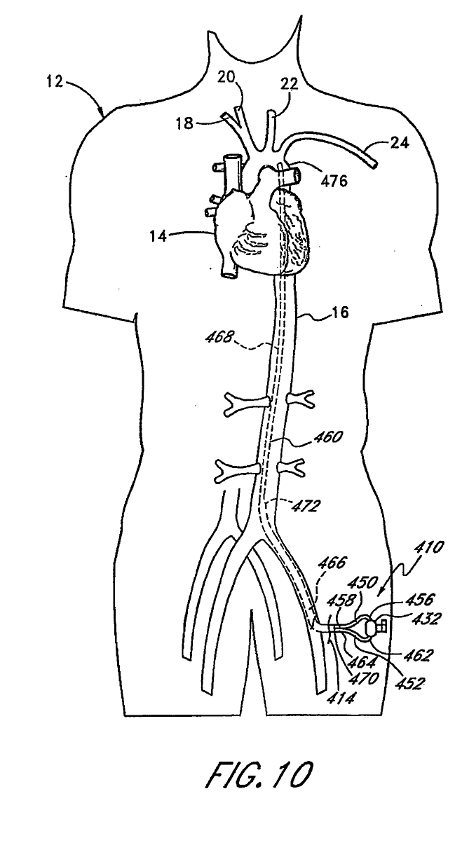

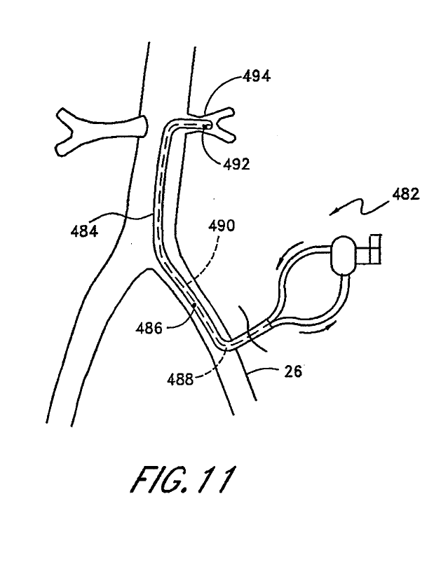

FIGS. 10 and 11 illustrate extracardiac heart assist systems that employ an extravascular pump and that can be applied through as a single-site system. FIG. 10 shows a system 410 that is applied to a patient 12 through a single cannulation site 414 while inflow and outflow conduits fluidly communicate with non-primary vessels. The heart assist system 410 is applied to the patient 12 percutaneously through a single site to couple two blood vessels with a pump 432. The pump 432 can have any of the features described in connection the pump 32. The pump 432 has an inflow conduit 450 and an outflow conduit 452 associated therewith. The inflow conduit 450 has a first end 456 and a second end 458. The first end 456 of the inflow conduit 450 is connected to the inlet of the pump 432 and the second end 458 of the inflow conduit 450 is fluidly coupled with a first non-primary blood vessel (e.g., the femoral artery 26) by way of a multilumen cannula 460. Similarly, the outflow conduit 452 has a first end 462 and a second end 464. The first end 462 of the outflow conduit 452 is connected to the outlet of the pump 432 and the second end 464 of the outflow conduit 452 is fluidly coupled with a second blood vessel (e.g., the descending aorta 16) by way of the multilumen cannula 460.

In one embodiment, the multilumen cannula 460 includes a first lumen 466 and a second lumen 468. The first lumen 466 extends from a proximal end 470 of the multilumen cannula 460 to a first distal end 472. The second lumen 468 extends from the proximal end 470 to a second distal end 474. In the illustrated embodiment, the second end 458 of the inflow conduit 450 is connected to the first lumen 466 of the multilumen cannula 460 and the second end 464 of the outflow conduit 452 is connected to the second lumen 468 of the multilumen cannula 460.

Where there is a desire for the patient 12 to be ambulatory, the multilumen cannula 460 preferably is made of material sufficiently flexible and resilient to permit the patient 12 to be comfortably move about while the multilumen cannula 460 is indwelling in the patient's blood vessels without causing any vascular trauma.

The application shown in FIG. 10 and described above results in flow from the first distal end 472 to the second distal end 474. Of course, the flow direction may be reversed using the same arrangement, resulting in flow from the distal end 474 to the distal end 472. In some applications, the system 410 is applied in an arterial-arterial fashion. For example, as illustrated, the multilumen cannula 460 can be inserted into the left femoral artery 26 of the patient 12 and guided superiorly through the descending aorta to one of numerous locations. In one application, the multilumen cannula 460 can be advanced until the distal end 474 is located in the aortic arch 476 of the patient 12. The blood could discharge, for example, directly into the descending aorta proximate an arterial branch, such as the left subclavian artery or directly into the peripheral mesenteric artery (not shown).

The pump 432 draws blood from the patient's vascular system in the area near the distal end 472 and into the lumen 466. This blood is further drawn into the lumen of the conduit 450 and into the pump 432. The pump 432 then expels the blood into the lumen of the outflow conduit 452, which carries the blood into the lumen 468 of the multilumen cannula 460 and back into the patient's vascular system in the area near the distal end 474.

FIG. 11 shows another embodiment of a heart assist system 482 that is similar to the heart assist system 410, except as set forth below. The system 482 employs a multilumen cannula 484. In one application, the multilumen cannula 484 is inserted into the left femoral artery 26 and guided superiorly through the descending aorta to one of numerous locations. Preferably, the multilumen cannula 484 has an inflow port 486 that is positioned in one application within the left femoral artery 26 when the cannula 484 is fully inserted so that blood drawn from the left femoral artery 26 is directed through the inflow port 486 into a first lumen 488 in the cannula 484. The inflow port 486 can also be positioned in any other suitable location within the vasculature, described herein or apparent to one skilled in the art. This blood is then pumped through a second lumen 490 in the cannula 484 and out through an outflow port 492 at the distal end of the cannula 484. The outflow port 492 may be situated within, for example, a mesenteric artery 494 such that blood flow results from the left femoral artery 26 to the mesenteric artery 494. The blood could discharge, for example, directly into the descending aorta proximate an arterial branch, such as the renal arteries, the left subclavian artery, or directly into the peripheral mesenteric artery 494, as illustrated in FIG. 11. Where there is a desire for the patient to be ambulatory, the multilumen cannula 484 preferably is made of material sufficiently flexible and resilient to permit the patient 12 to comfortably move about while the cannula 484 is indwelling in the patient's blood vessels without causing any vascular trauma.

Further details of the multilumen cannula 460 are described below in connection with FIG. 11, and in U.S. patent application Ser. No. 10/078,283, which is incorporated by reference hereinabove.

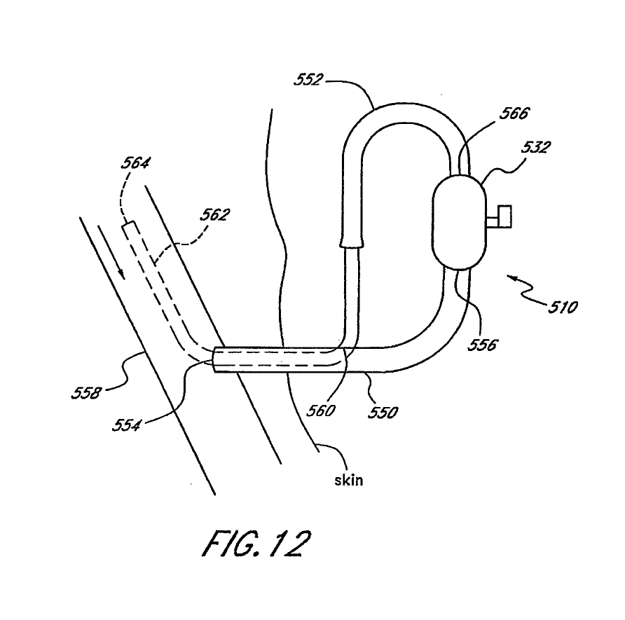

FIG. 12 shows another heart assist system 510 that takes further advantage of the supplemental blood perfusion and heart load reduction benefits while remaining minimally invasive in application. The heart assist system 510 is an extracardiac pumping system that includes a pump 532, an inflow conduit 550 and an outflow conduit 552. In the illustrated embodiment, the inflow conduit 550 comprises a vascular graft. The vascular graft conduit 550 and the outflow conduit 552 are fluidly coupled to pump 532. The pump 532 is configured to pump blood through the patient at subcardiac volumetric rates, and has an average flow rate that, during normal operation thereof, is substantially below that of the patient's heart when healthy. In one variation, the pump 532 may be a rotary pump. Other pumps described herein, or any other suitable pump can also be used in the extracardiac pumping system 510. In one application, the pump 532 is configured so as to be implantable.

The vascular graft 550 has a first end 554 and a second end 556. The first end 554 is sized and configured to couple to a non-primary blood vessel 558 subcutaneously to permit application of the extracardiac pumping system 510 in a minimally-invasive procedure. In one application, the vascular graft conduit 550 is configured to couple to the blood vessel 558 via an anastomosis connection. The second end 556 of the vascular graft 550 is fluidly coupled to the pump 532 to conduct blood between the non-primary blood vessel 558 and the pump 532. In the embodiment shown, the second end 556 is directly connected to the pump 532, but, as discussed above in connection with other embodiments, intervening fluid conducting elements may be interposed between the second end 556 of the vascular graft 550 and the pump 532. Examples of arrangements of vascular graft conduits may be found in U.S. application Ser. No. 09/780,083, filed Feb. 9, 2001, entitled EXTRA-CORPOREAL VASCULAR CONDUIT, which is hereby incorporated by reference in its entirety and made a part of this specification.

FIG. 12 illustrates that the present inventive embodiment further comprises means for coupling the outflow conduit 552 to the vascular graft 550, which may comprise in one embodiment an insertion site 560. In the illustrated embodiment, the insertion site 560 is located between the first end 554 and the second end 556 of the vascular graft 550. The outflow conduit 552 preferably is coupled with a cannula 562. The cannula 562 preferably takes any suitable form and may be modified as suggested in U.S. patent application Ser. No. 10/078,283, incorporated by reference hereinabove.

The insertion site 560 is configured to receive the cannula 562 therethrough in a sealable manner in the illustrated embodiment. In another embodiment, the insertion site 560 is configured to receive the outflow conduit 552 directly. The cannula 562 includes a first end 564 sized and configured to be inserted through the insertion site 560, through the cannula 550, and through the non-primary blood vessel 558. The conduit 552 has a second end 566 fluidly coupled to the pump 532 to conduct blood between the pump 532 and the blood vessel 558.

The extracardiac pumping system 510 can be applied to a patient, as shown in FIG. 12, so that the outflow conduit 552 provides fluid communication between the pump 532 and a location upstream or downstream of the point where the cannula 562 enters the non-primary blood vessel 558. In another application, the cannula 562 is directed through the blood vessel to a different blood vessel, upstream or downstream thereof. Although the vascular graft 550 is described above as an “inflow conduit” and the conduit 552 is described above as an “outflow conduit,” in another application of this embodiment, the blood flow through the pumping system 510 is reversed (i.e., the pump 532 pumps blood in the opposite direction), whereby the vascular graft 550 is an outflow conduit and the conduit 552 is an inflow conduit.

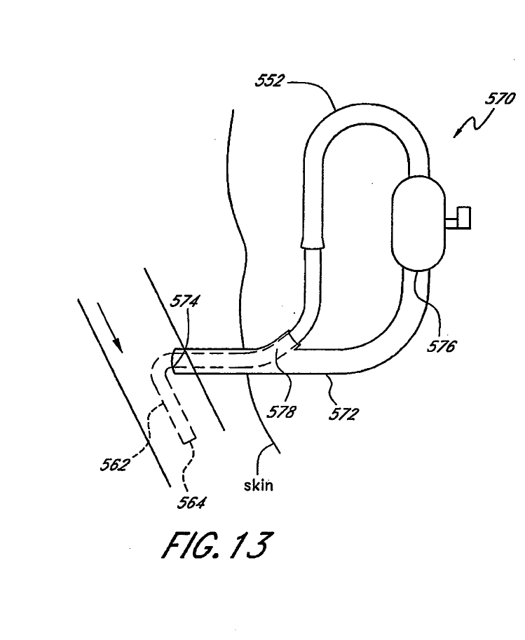

FIG. 13 shows a variation of the extracardiac pumping system shown in FIG. 12. In particular, a heart assist system 570 includes an inflow conduit 572 that comprises a first end 574, a second end 576, and means for connecting the outflow conduit 552 to the inflow conduit 572. In one embodiment, the inflow conduit 572 comprises a vascular graft. The extracardiac pumping system 570 is otherwise similar to the extracardiac pumping system 510. The means for connecting the conduit 552 to the inflow conduit 572 may comprise a branched portion 578. In one embodiment, the branched portion 578 is located between the first end 574 and the second end 576. The branched portion 578 is configured to sealably receive the distal end 564 of the outflow conduit 552. Where, as shown, the first end 564 of the outflow conduit 552 comprises the cannula 562, the branched portion 578 is configured to receive the cannula 562. The inflow conduit 572 of this arrangement comprises in part a multilumen cannula, where the internal lumen extends into the blood vessel 558. Other multilumen catheter arrangements are shown in U.S. application Ser. No. 10/078,283, incorporated by reference herein above.

2. Single-Site Application of Intravascular Pumping Systems

FIG. 14-20 illustrate extracardiac heart assist systems that employ intravascular pumping systems. Such systems take further advantage of the supplemental blood perfusion and heart load reduction benefits discussed above while remaining minimally invasive in application. Specifically, it is contemplated to provide an extracardiac pumping system that comprises a pump that is sized and configured to be at least partially implanted intravascularly in any location desirable to achieve those benefits, while being insertable through a non-primary vessel.

FIG. 14 shows a heart assist system 612 that includes a pumping means 614 comprising preferably one or more rotatable impeller blades 616, although other types of pumping means 614 are contemplated, such as an Archimedes screw, a worm pump, or other means by which blood may be directed axially along the pumping means from a point upstream of an inlet to the pumping means to a point downstream of an outlet from the pumping means. Where one or more impeller blades 616 are used, such as in a rotary pump, such impeller blades 616 may be supported helically or otherwise on a shaft 618 within a housing 620. The housing 620 may be open, as shown, in which the walls of the housing 620 are open to blood flow therethrough. The housing 620 may be entirely closed, if desired, except for an inlet and outlet (not shown) to permit blood flow therethrough in a more channel fashion. For example, the housing 620 could be coupled with or replaced by a cannula with a redirecting tip portion, as suggested by U.S. patent application Ser. No. 10/078,283, incorporated by reference hereinabove. The heart assist system 612 serves to supplement the kinetic energy of the blood flow through the blood vessel in which the pump is positioned, e.g., the aorta 16.

The impeller blade(s) 616 of the pumping means 614 of this embodiment may be driven in one or a number of ways known to persons of ordinary skill in the art. In the embodiment shown in FIG. 14, the impeller blade(s) 616 are driven mechanically via a rotatable cable or drive wire 622 by driving means 624, the latter of which may be positioned corporeally (intra- or extra-vascularly) or extracorporeally. As shown, the driving means 624 may comprise a motor 626 to which energy is supplied directly via an associated battery or an external power source, in a manner described in more detail herein. It is also contemplated that the impeller blade(s) 616 be driven electromagnetically through an internal or external electromagnetic drive. Preferably, a controller (not shown) is provided in association with this embodiment so that the pumping means 614 may be controlled to operate in a continuous and/or pulsatile fashion, as described herein.

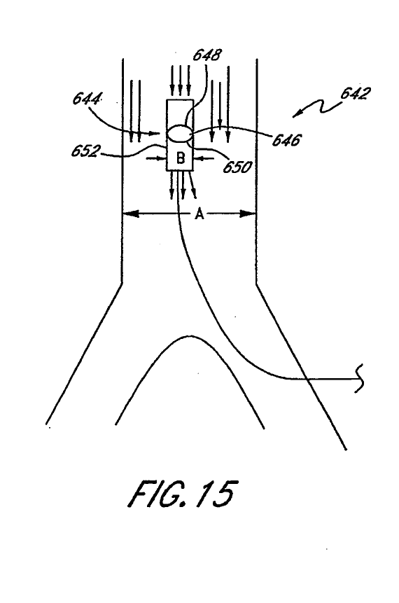

Variations of the intravascular embodiment of FIG. 14 are shown in FIGS. 15 and 16. In the embodiment of FIG. 15, an intravascular extracardiac system 642 comprising a pumping means 644, which may be one of several means described herein. The pumping means 644 may be driven in any suitable manner, including means sized and configured to be implantable and, if desired, implantable intravascularly, e.g., as discussed above. For a blood vessel (e.g., descending aorta) having a diameter “A”, the pumping means 644 preferably has a meaningfully smaller diameter “B”. The pumping means 644 may comprise a pump 646 having an inlet 648 and an outlet 650. The pumping means 644 also comprises a pump driven mechanically by a suitable drive arrangement in one embodiment. Although the vertical arrows in FIG. 15 illustrate that the pumping means 644 pumps blood in the same direction as the flow of blood in the vessel, the pumping means 644 could be reversed to pump blood in a direction generally opposite of the flow in the vessel.

In one embodiment, the pumping means 644 also includes a conduit 652 in which the pump 646 is housed. The conduit 652 may be relatively short, as shown, or may extend well within the designated blood vessel or even into an adjoining or remote blood vessel at either the inlet end, the outlet end, or both. The intravascular extracardiac system 642 may further comprise an additional parallel-flow conduit, as discussed below in connection with the system of FIG. 16.

The intravascular extracardiac system 642 may further comprise inflow and/or outflow conduits or cannulae (not shown) fluidly connected to the pumping means 644, e.g., to the inlet and outlet of pump 646. Any suitable conduit or cannula can be employed.

In another embodiment, an intravascular pumping means 644 may be positioned within one lumen of a multilumen catheter so that, for example, where the catheter is applied at the left femoral artery, a first lumen may extend into the aorta proximate the left subclavian and the pumping means may reside at any point within the first lumen, and the second lumen may extend much shorter just into the left femoral or left iliac. Such a system is described in greater detail in U.S. application Ser. No. 10/078,283, incorporated by reference herein above.

FIG. 16 shows a variation of the heart assist system of FIG. 15. In particular the intravascular system may further comprise an additional conduit 660 positioned preferably proximate the pumping means 644 to provide a defined flow path for blood flow axially parallel to the blood flowing through the pumping means 644. In the case of the pumping means 644 of FIG. 16, the means comprises a rotatable cable 662 having blood directing means 664 supported therein for directing blood axially along the cable. Other types of pumping means are also contemplated, if desired, for use with the additional conduit 660.

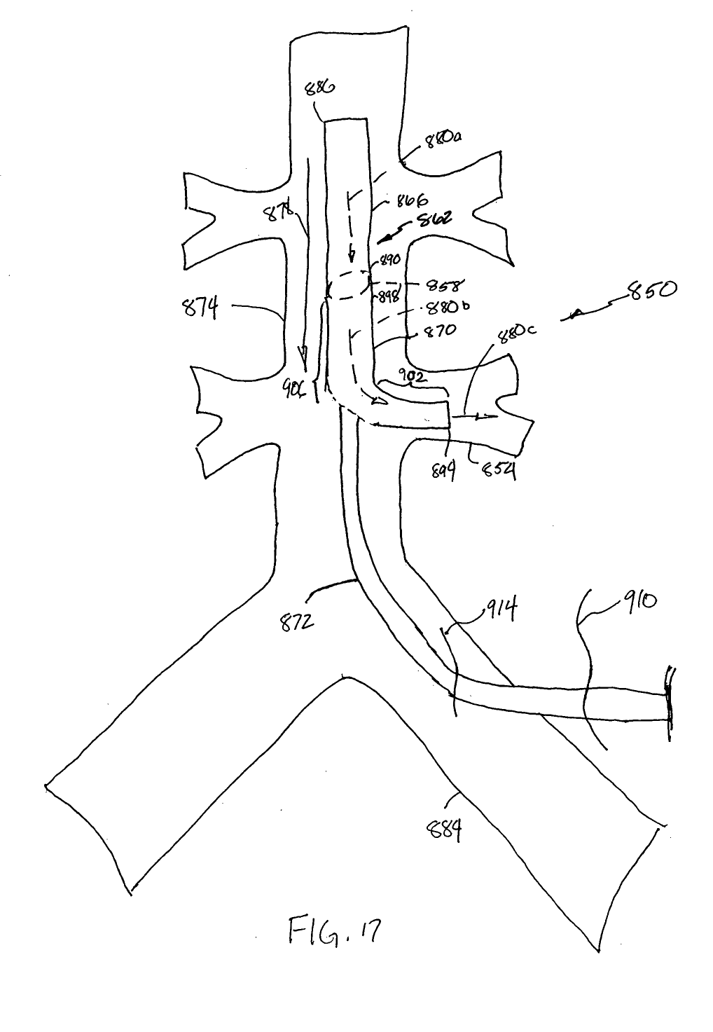

Further variations of intravascular extracardiac pumping systems, examples of which are shown in FIGS. 17-20, are arranged to increase perfusion to specific tissues, e.g., particular organs, through branch blood vessels. FIG. 17 shows an intravascular extracardiac pumping system 850 that may be applied to pump blood in a manner that increases perfusion of specific tissues, e.g., particular organs, through a branch artery, e.g., the left renal artery 854, without any component of the pumping system 850 being connected to the patient's heart to address the demands of the kidneys that decrease the ability of the ailing heart to heal. The system 850 includes a pump 858 and a pump housing 862. As discussed more fully below, the pump housing 862 provides an inflow portion 866, an outflow portion 870, and a positioning means 872. The pump housing 862 houses the pump 858 and also enables the system 850 to be positioned within the vasculature, as discussed more fully below.

The pump 858 may operate in a manner similar to the pumps described hereinabove. For example, the pump 858 may be configured to pump blood through the patient at subcardiac volumetric rates. During normal operation of the pump 858, the volumetric rate of the blood pumped through the pump 858 is substantially below that of the patient's heart when healthy, as discussed above. The pump 858 is configured to be positioned within the patient's vasculature. The pump 858 may be any suitable pump or pumping means. For example, the pump 858 may be configured as a rotary pump, an impeller, an Archimedes screw, or any other suitable pump arrangement. The pump 858 may comprise a rotatable cable having means for directing blood axially along the cable. The pump 858 may be a helically shaped pump or pumping means, as discussed above.

The pump 858 may be operated in a continuous flow, pulsatile, or other desirable mode. The pump 858 may be driven through any suitable pump driving means, e.g., a drive wire or cable (as in FIG. 16), a motor, electromagnetically by a discrete electromagnetic drive, or in any other suitable means. The pump driving means preferably is configured to be implantable. Where an electromagnetic drive is employed, it is preferably sized and configured to be implantable beneath the skin of the patient, e.g., in the patient's vasculature.

The intravascular extracardiac pumping system 850 preferably is configured so that the pump 858 may be positioned at a variety of locations within the vasculature, e.g., within the aorta 874, when applied. In one application of the pumping system 850, the pump 858 resides in the aorta 874 midstream the heart of the patient and the left renal artery 854. As used herein “midstream” is a broad term that includes a location closer to one end or the other of a vascular portion (e.g., a location within the aorta closer to a renal artery than to an iliac artery or closer to an iliac artery than to a renal artery), as well as about half-way between two ends of the vascular portion. The direction of the natural vascular flow of blood in the aorta is indicated by an arrow 878. In this application, the pumping system 850 provides parallel flow to increase perfusion of tissues, e.g., the pump 858 directs blood in generally the same direction as the flow of blood in the aorta 874 outside the system 850 (indicated by the arrow 878). The flow of blood through the system 850 is indicated by arrows 880, discussed further below. This flow direction is preferable where the pump 858 is located midstream the heart and the renal artery 854, as in the application of FIG. 17.

In another application, the system 850 is applied to the patient such that the pump 858 resides or is positioned midstream the renal artery and a femoral/iliac artery 884 of the patient. When applied in this manner, the pump 858 preferably is configured to direct blood generally counter to the direction of the blood flowing in the aorta 874 adjacent the pump 858 and outside the pumping system.850 (generally counter to the direction of the arrow 878). As discussed more fully below, the pump 858 can be operated to deliver blood to the renal artery 854 (or other branch artery at a vascular location between the femoral arteries and the heart) where it is inserted into an artery above the renal artery (or other branch artery), e.g., through the subclavian or axillary artery, and advanced down the aorta to position the pump 858 midstream the renal artery 854 and the femoral/iliac artery 884.

In one embodiment, the pump 858 is reversible such that the flow of blood through the system 850 may be either generally in the same direction or generally counter to the direction of blood-flow outside the system 850 in the blood vessel in which the pump 858 resides. This arrangement advantageously would allow the physician to determine during the procedure how far the pump 858 is to be advanced into the vasculature.

The inflow portion 866 extends between a first end 886 and a second end 890. The second end 890 is coupled with the pump 858. The first end 886 is remote from the pump 854 and generally acts as an inlet end in some applications. The inflow portion 866 is fluidly coupled to the pump 858 to convey blood to the pump 858 from a location midstream the pump 858 and the heart, e.g., upstream of the renal artery 854 in the aorta in the application of FIG. 17. In the illustrated application, the first end 886 is positioned just above the mesenteric artery. In another application, the first end 886 of the inflow portion 866 is configured to extend to a vascular location proximate the aortic arch of the patient (e.g., as in FIG. 7). The inflow portion 866 may be positioned in any desired location within the vasculature. A variety of tip arrangements may be provided at the first end 886 of the inflow portion 866, including those set forth in U.S. patent application Ser. No. 10/078,283, filed Feb. 14, 2002, which is hereby incorporated by reference herein in its entirety. In various applications, the inflow portion 866 is positioned within the vasculature of the patient by the positioning means 872, as discussed more fully below.

The outflow portion 870 extends between a first end 894 and a second end 898. The first end 894 is remote from the pump 858 and will generally operate as the outlet end. The second end 898 is coupled with the pump 858. The outflow portion 870 directs blood away from the pump 858, preferably to a branch artery, e.g., the renal artery 854, when applied. In another embodiment, the outflow portion 870 directs blood to a vascular location in the aorta more remote from the heart than is the pump 858. In another embodiment, the outflow portion 870 directs blood to a location in the aorta 874 midstream the heart and the pump 858. In one advantageous application, the pumping system 850 is applied so that the first end 894 of the outflow portion 870 is positioned within the renal artery 854 of the patient. When the pumping system 850 is applied in this manner and operated, blood perfusion to tissues through the renal artery 854, e.g., to the kidneys, is increased. Although shown applied to the left renal artery, the pumping system 850 could also be applied to the right renal artery.

In one embodiment, the outflow portion 870 comprises a first portion 902 and a second portion 906. The second portion 906 of the outflow conduit 870 includes the second end 898 of the outflow portion 870 and extend proximally from the pump 858. The first portion 902 of the outflow portion 870 extends laterally from the second portion 906 of the outflow conduit 870 when applied. In one arrangement, shown in FIG. 17, the first portion 902 of the outflow conduit 870 extends generally perpendicularly to the second portion 906 of the outflow conduit 870. In this arrangement, the outflow portion 870 can be said to have a generally L-shaped configuration. In one application, the pumping system 850 is applied to the patient such that the second portion 906 extends to a location adjacent to the renal artery 854 and the first portion 902 extends laterally into the renal artery 854.

The arrangement of the first portion 902 with respect to the second portion 906 may be varied as desired. For example, the first and second portions 902, 906 may form any suitable angle therebetween. Also, the first portion 902 may be configured to deliver blood in a suitable manner, e.g., by having a length sufficient to position the first end 894 in a selected sub-branch of the branch artery in which the first portion 902 resides, by defining an angle with the second portion 906 selected to direct blood out of the first end 894 toward a selected sub-branch, or by having an outflow tip, e.g., those described in application Ser. No. 10/078,283, incorporated by reference hereinabove.

The pumping system 850 may be applied in any suitable manner, e.g., by way of open surgery or minimally invasively. In one embodiment, the positioning means 872 is provided to enable the minimally invasive application of the pumping system 850. In one embodiment, the positioning means 872 is an elongate body that extends generally proximally from the pump 858. The positioning means 872 preferably is configured such that the pumping system 850 may be inserted subcutaneously into the vasculature in a minimally-invasive procedure. In one embodiment, the positioning means 872 is a generally low-profile structure, e.g., having a relatively small cross-sectional profile, and has sufficient axial stiffness to allow the pumping system 850 to be advanced through the skin, at a percutaneous insertion site indicated by a line 910. The positioning means 872 further enables the system 850 to be advanced through tissue between the insertion site 910 and though the wall of a target vessel of the vasculature into the target vessel, as indicted by a line 914, and into a selected position, e.g., as shown in FIG. 17. In one embodiment, the positioning means 872 resembles a catheter and may have a lumen through which a portion of the pump 858, e.g., a drivewire, may extend.

Although the first and second portions 902, 906 are formed generally perpendicular to each other, the pumping system 850 preferably is configured to be applied in a minimally invasive manner. For example, the first portion 902 may be deformable with respect to second portion 906 to lower the profile of the outflow portion 870. In one embodiment, the first portion 902 has very low stiffness and is collapsible against the outer wall of the positioning means 872 during insertion or withdrawal. In another embodiment, a sleeve (not shown) may extend over the outer wall of the positioning means 872 at least far enough distally to cover the first portion 902 during advancement or withdrawal of the pumping system 850 to maintain the low cross-sectional profile thereof. 101071 Although the pumping system 850 is illustrated as being applied through the left femoral/iliac artery, the system 850 could applied in open surgery or minimally invasively by way of a different peripheral vessel, e.g., the right femoral artery, the left femoral artery, the right iliac artery, the left iliac artery, the left subclavian artery, the right axillary artery, or any other suitable peripheral vessel. When applied from above (e.g., from the subclavian or axillary), the system 850 may be advanced until the inlet end 886 is at a location more remote from the heart than the selected branch artery, e.g., at a location downstream of a selected branch artery, e.g. the renal artery 854. From this location, the pumping system 850 can direct blood generally counter to the direction of flow in the aorta 874 outside the pumping system 850 into a branch artery, such as the renal artery 854. In one application, the pumping system 850 is advanced down the aorta until the inlet end 886 is in an iliac artery and/or a femoral artery. In another application, the pumping system 850 is advanced down the aorta until the inlet end 886 is adjacent the iliac bifurcation.

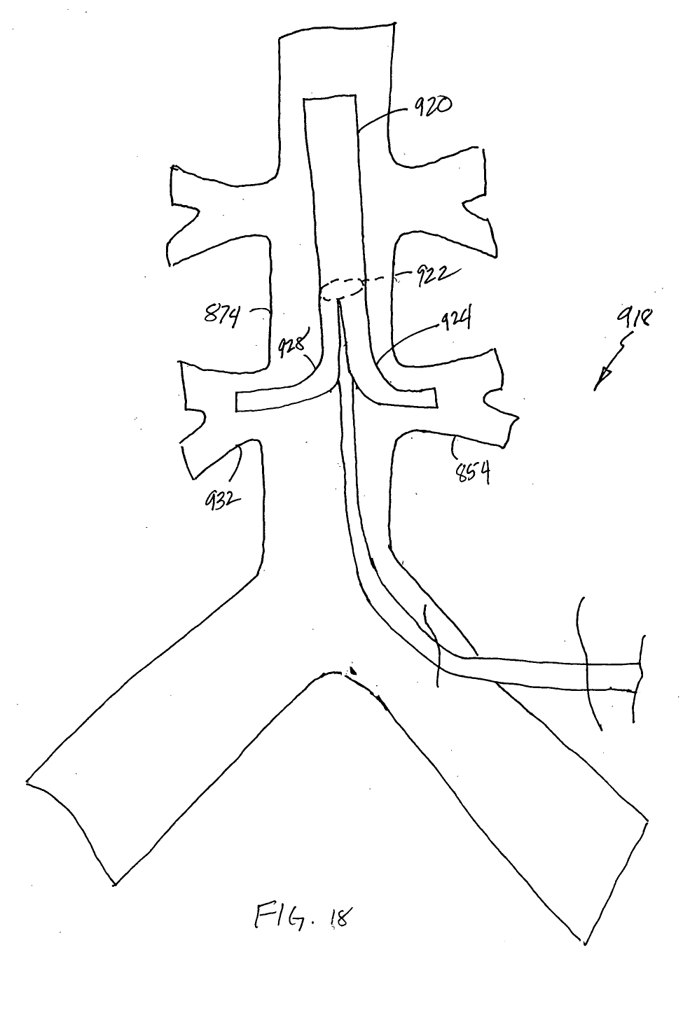

081 FIG. 18 illustrates an intravascular extracardiac pumping system 918 that is similar to the pumping system 850, except as set forth below. In particular, the pumping system 918 includes an inflow portion 920 and a pump 922 that is fluidly coupled with a first outflow portion 924 and a second outflow portion 928. The first outflow portion 924 is similar to the outflow portion 870 and is configured to be positioned within a branch artery, e.g., the left renal artery 854, when applied to the patient.

The second outflow portion 928 is configured to be positioned within a vessel of the patient. In particular, the second outflow portion 928 may be positioned within a renal artery, e.g., the right renal artery 932 as shown in FIG. 18. Although shown in the right renal artery 932, the second outflow portion 928 may be positioned in another artery, e.g., in the aorta 874, in the right or left iliac artery, the right or left femoral artery, or in any other branch artery, as discussed above.

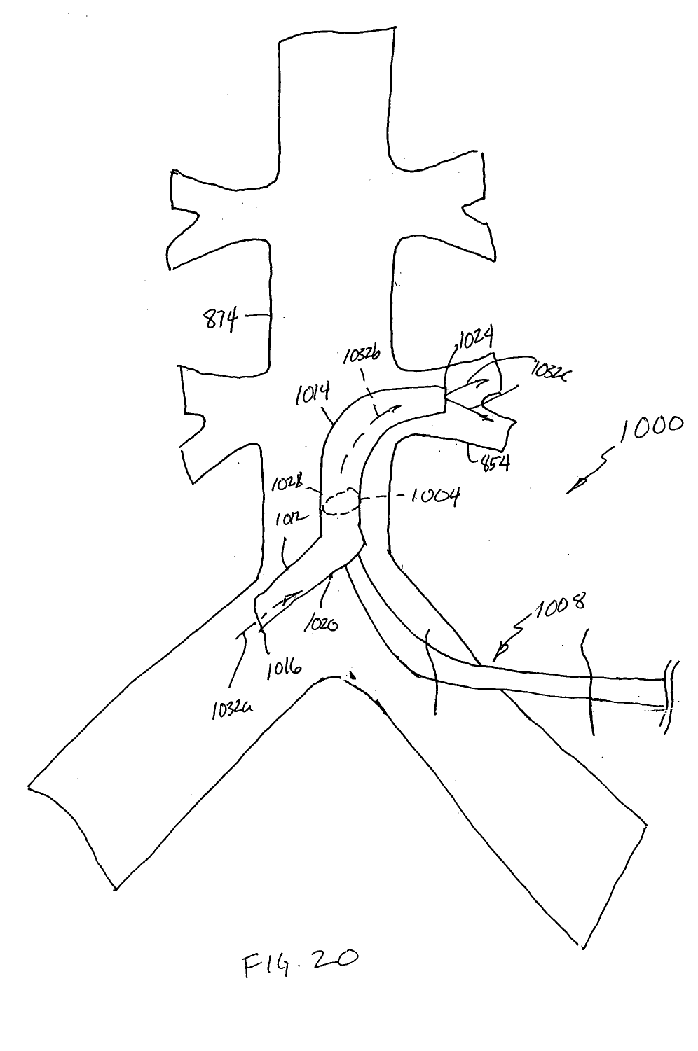

As discussed in connection with the outflow portion 870 of the pumping system 850, the first outflow portion 924 and the second outflow portion 928 may be configured to extend from the pump 922 to a vascular location adjacent the left and right renal arteries 854, 932 and from that vascular location into arteries 854, 932. In one arrangement at one of the first and second outflow portions 924, 928 forms a generally L-shaped configuration whereby the outlet ends thereof extend into the left and right renal arteries 854, 932. FIG. 18 shows that both the first and second outflow portions 924, 928 could form generally L-shaped configuration in some embodiments. As discussed above in connection with the system 850, the system 918 can be applied through an upper body peripheral vessel and advanced down the aorta until the inlet end of the inflow portion 920 is in an iliac artery and/or a femoral artery. In another application, the pumping system 918 is advanced down the aorta until the inlet end is adjacent the iliac bifurcation. Also, the system 918 could be modified in a manner similar to the modification of the system 850, which is shown in FIG. 20 as the pumping system 1000, e.g., providing a system that has an inflow portion between a pump and a proximal end and providing two or more outflow portions between the pump and a distal end, which system may be configured to be applied through a lower body peripheral vessel.

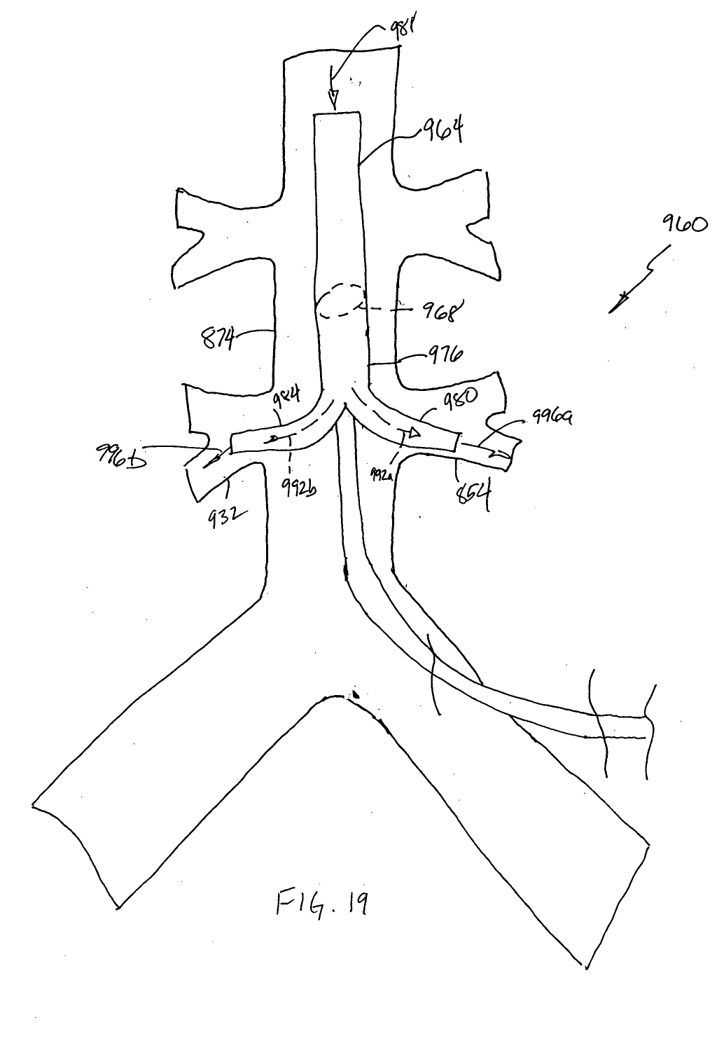

FIG. 19 illustrates another embodiment of an intravascular extracardiac pumping system 960. The system 960 is similar to the system 920 except as set forth below. The pumping system 960 has an inflow portion 964, a pump 968, and a Y-shaped outflow portion 972. The Y-shaped outflow portion 972 includes a first portion 976, a second portion 980, and a third portion 984. The first portion 976 extends proximally from the pump 968. In one application, the first portion 976 extends to a location adjacent the left and right renal arteries 854, 932. The second portion 980 of the Y-shaped outflow portion 972 extends laterally from the first portion 976. The third portion 984 extends laterally from the Y-shaped portion 972 on an opposite side of the Y-shaped portion 972 from the second portion 980. In one application, the second and third portions 980, 984 extend from adjacent the left and right renal arteries 854, 932 into the left and right renal arteries 854, 932 respectively. Blood drawn into the inflow portion 964, indicated by an arrow 988, is directed by the pump 968 into the first and second portions 980, 984 of the Y-shaped outflow portion 972, as indicated by arrows 992a, 992b, and is directed thereby into one or more regions of the renal arteries 854, 932, as indicated by arrows 996a, 996b. The pumping system 960 could also be applied to direct blood into other blood vessels, e.g., into the right and/or left femoral arteries, into the right and/or left iliac arteries, or into any other branch artery(ies) or vessel(s) or portions of branch arteries or vessels. The system 960 could be modified in a manner similar to the modification of the system 850, which is shown in FIG. 20 as the pumping system 1000, e.g., providing a system that has an inflow portion between a pump and a proximal end and providing two or more outflow portions between the pump and a distal end, which system may be configured to be applied through a lower body peripheral vessel.

The systems 850, 918, and 960 can be applied, as shown in FIGS. 17-19, such that an inlet end is located midstream a pump and the heart of the patient. As discussed above, the embodiments applied in FIGS. 17-19 could be applied such that the pump is located midstream the inlet end and the heart, e.g., by inserting the systems of FIGS. 17-19 percutaneously and minimally invasively through the subclavian or other peripheral vessel and down the aorta. The embodiments of FIGS. 17-19 can also be modified such that when applied through a peripheral vessel below the aorta, the pump is between the inlet end and the heart.

FIG. 20 shows an intravascular extracardiac pumping system 1000 that includes a pump 1004 and a pump housing 1008. The pump 1004 is similar to the pump 858. The pump housing 1008 includes an inflow portion 1012 and an outflow portion 1014. The pump housing 1008 houses the pump 1004 and positioned the pumping system 1000 within the vasculature in a manner similar to the pump housing 862. In one application, the pumping system 1000 is positioned within the vasculature such that the pump 1004 resides at a location midstream a femoral artery and the heart.

The inflow portion 1012 extends between a first end 1016 and a second end 1020. The second end 1020 is coupled with the pump 1004. The first end 1016 is remote from the pump 1004 and operates as an inlet end in some applications. The first end 1016 is fluidly coupled to the pump 1004 to convey blood to the pump 1004 from a location more remote from the heart than is the pump 1004, e.g., downstream of the pump 1004 in the aorta or in a femoral artery, as shown. The inflow portion 1012 may be positioned in any desired location within the vasculature. In the illustrated application, pumping system 1000 is applied such that the first end 1016 of the inflow portion 1012 extends into an upper portion of the right femoral/iliac artery. Other possible applications include applying the pumping system 1000 such that the first end 1016 of the inflow portion 1012 extends into a portion of the right or left femoral artery, the right or left iliac artery, or into the abdominal aorta, e.g. just above the iliac bifurcation. A variety of tip arrangements, such as those set forth in application Ser. No. 10/078,283, incorporated by reference hereinabove, may be provided at the first end 1016 of the inflow portion 1012.

The outflow portion 1014 extends between a first end 1024 and a second end 1028. The first end 1024 is remote from the pump 1004 and will generally operate as the outlet end in application. The second end 1028 is coupled with the pump 1004. The outflow portion 1014 directs blood away from the pump 1004, preferably to a branch artery, e.g., the renal artery 854, when applied. In another application, the outflow portion 1014 directs blood to a location in the aorta 874 midstream the pump 1004 and the heart. In one advantageous application, the pumping system 1000 is applied so that the first end 1024 of the outflow portion 1014 is positioned within the renal artery 854 of the patient. When the pumping system 1000 is applied in this manner and operated, blood perfusion to tissues through the renal artery 854, e.g., to the kidneys, is increased. Although shown applied to the left renal artery, the pumping system 1000 could also be applied to the right renal artery or other branch artery.

As discussed above in connection with the outflow portion 870, the outflow portion 1014 could form a generally L-shaped configuration. The outflow portion 1014 could also be modified to provide advantageous blood flow characteristics at the outlet end, e.g., by incorporating a tip arrangement such as those described in application Ser. No. 10/078,283. Variations similar to those shown in FIG. 20 may be applied to the embodiments of FIGS. 18-19 to provide pumping systems applicable through femoral arteries and other lower-body peripheral vessels that provide outflow portions at a location midstream a pump and the heart when applied.

In operation, the system 1000 directs blood through the pump housing 1008 generally counter to flow of blood in the vessel outside the system 1000. As discussed above, blood flow outside the system 1000 is illustrated by the arrow 878. Blood flow is drawn into the system 1000 in one application through the first end 1016, as indicated by an arrow 1032a. The pump 1004 then directs the blood into the outflow portion 1014, as indicated by an arrow 1032b. Blood is then directed by the system 1000 into the renal artery 854 through the outlet end 1024, as indicated by arrows 1032c.

As discussed above, the pumping systems described herein, particularly those described in connection with FIGS. 17-20, can be used to perform a variety of methods. One method which may be performed is a method for treating a patient without connecting any component to the patient's heart. The method will be discussed primarily in connection with the pumping system 850, but could be applied with other systems, e.g. the systems 918, 960, 1000. The inlet end 886 of the inflow portion 866 of the intravascular pumping system 850 is inserted into the vasculature of a patient using a minimally invasive surgical procedure. In particular, the inlet end 886 may be directed through a small incision in the skin of the patient, e.g., the percutaneous insertion site 910. As discussed above, the intravascular pumping system 850 has a pump 858 coupled with an inflow portion 866 and an outflow portion 870.

The intravascular pumping system 850 is advanced into the vascular system, though the vascular insertion site 914, e.g., in the femoral/iliac artery 884, until the inlet end 886 of the inflow portion 866 is positioned at a first location within a vessel. In FIG. 17, the first location is a location within the aorta 874. When at this position, the outlet end 894 of the outflow portion 870 preferably is positioned within a branch artery, e.g., the renal artery 854. As discussed above, the outflow portion 870 may be configured to enable minimally invasive application of the system 850. As discussed above, the positioning means 872 may be employed to advance the intravascular pumping system 850 to the desired location within the vasculature in a manner similar to the advancement of a catheter.

In one method, after the system 850 has been positioned, as discussed above, the pump 858 draws blood into the inflow portion 866 and toward the pump 858, as indicated by an arrow 880a. The pump 858 pumps blood into the outflow portion 870, as indicated by an arrow 880b. Thereafter, in the illustrated application, the blood in the outflow portion 870 is forced out into the left renal artery 854, as indicated by the arrow 880c, to perfuse tissue through the renal artery 854 at volumetric rates that are on average subcardiac. Although the method is discussed in connection with the system 850, which is shown applied to the left renal artery 854 in FIG. 15, other methods can employ the pumping system 850 to pump blood into other vessels, such as other arteries, including the right renal artery 932 and other branch arteries. Also, other methods can be performed using other pumping systems, e.g., the pumping systems 918, 960, 1000 to perfuse tissue through a branch artery.

C. Potential Enhancement of Systemic Arterial Blood Mixing

One of the advantages of the present invention is its potential to enhance mixing of systemic arterial blood, particularly in the aorta. Such enhanced mixing ensures the delivery of blood with higher oxygen-carrying capacity to organs supplied by arterial side branches off of the aorta. A method of enhancing mixing utilizing the present invention preferably includes taking steps to assess certain parameters of the patient and then to determine the minimum output of the pump that, when combined with the heart output, ensures turbulent flow in the aorta, thereby enhancing blood mixing.

Blood flow in the aortic arch during normal cardiac output may be characterized as turbulent in the end systolic phase. It is known that turbulence in a flow of fluid through pipes and vessels enhances the uniform distribution of particles within the fluid. It is believed that turbulence in the descending aorta enhances the homogeneity of blood cell distribution in the aorta. It is also known that laminar flow of viscous fluids leads to a higher concentration of particulate in the central portion of pipes and vessels through which the fluid flows. It is believed that, in low flow states such as that experienced during heart failure, there is reduced or inadequate mixing of blood cells leading to a lower concentration of nutrients at the branches of the aorta to peripheral organs and tissues. As a result, the blood flowing into branch arteries off of the aorta will likely have a lower hematocrit, especially that flowing into the renal arteries, the celiac trunk, the spinal arteries, and the superior and inferior mesenteric arteries. That is because these branches draw from the periphery of the aorta The net effect of this phenomenon is that the blood flowing into these branch arteries has a lower oxygen-carrying capacity, because oxygen-carrying capacity is directly proportional to both hematocrit and the fractional O₂ saturation of hemoglobin. Under those circumstances, it is very possible that these organs will experience ischemia-related pathology.

The phenomenon of blood streaming in the aorta, and the resultant inadequate mixing of blood resulting in central lumenal concentration of blood cells, is believed to occur when the Reynolds number (N_{R}) for the blood flow in the aorta is below 2300. To help ensure that adequate mixing of blood will occur in the aorta to prevent blood cells from concentrating in the center of the lumen, a method of applying the present invention to a patient may also include steps to adjust the output of the pump to attain turbulent flow within the descending aorta upstream of the organ branches; i.e., flow exhibiting a peak Reynolds number of at least 2300 within a complete cycle of systole and diastole. Because flow through a patient is pulsatile in nature, and not continuous, consideration must be given to how frequently the blood flow through the aorta has reached a certain desired velocity and, thus, a desired Reynolds number. The method contemplated herein, therefore, should also include the step of calculating the average Womersley number (N_{W}), which is a function of the frequency of the patient's heart beat. It is desired that a peak Reynolds number of at least 2300 is attained when the corresponding Womersley number for the same blood flow is approximately 6 or above.

More specifically, the method may comprise calculating the Reynolds number for the blood flow in the descending aorta by determining the blood vessel diameter and both the velocity and viscosity of the fluid flowing through the aorta. The Reynolds number may be calculated pursuant to the following equation:

$${N}_{R} = \frac{V \cdot d}{υ}$$

where: V=the velocity of the fluid; d=the diameter of the vessel; and υ=the viscosity of the fluid. The velocity of the blood flowing through the aorta is a function of the cross-sectional area of the aorta and the volume of flow therethrough, the latter of which is contributed both by the patient's own cardiac output and by the output of the pump of the present invention. Velocity may be calculated by the following equation:

$$V = \frac{Q}{π {r}^{2}}$$

where Q=the volume of blood flowing through the blood vessel per unit time, e. g., the aorta, and r=radius of the aorta. If the relationship between the pump output and the velocity is already known or independently determinable, the volume of blood flow Q may consist only of the patient's cardiac output, with the knowledge that that output will be supplemented by the subcardiac pump that is part of the present invention. If desired, however, the present system can be implemented and applied to the patient first, before calculating Q, which would consist of the combination of cardiac output and the pump output.

The Womersley number may be calculated as follows:
N_{W}=r\sqrt{2πΩ/υ}
where r is the radius of the vessel being assessed, Ω is the frequency of the patient's heartbeat, and υ=the viscosity of the fluid. For a peak Reynolds number of at least 2300, a Womersley number of at least 6 is preferred, although a value as low as 5 would be acceptable.

By determining (i) the viscosity of the patient's blood, which is normally about 3.0 mm²/sec (kinematic viscosity), (ii) the cardiac output of the patient, which of course varies depending upon the level of CHF and activity, and (iii) the diameter of the patient's descending aorta, which varies from patient to patient but is about 21 mm for an average adult, one can determine the flow rate Q that would result in a velocity through the aorta necessary to attain a Reynolds number of at least 2300 at its peak during the patient's heart cycle. Based upon that determination of Q, one may adjust the output of the pump of the present invention to attain the desired turbulent flow characteristic through the aorta, enhancing mixing of the blood therethrough.

One may use ultrasound (e.g., echocardiography or abdominal ultrasound) to measure the diameter of the aorta, which is relatively uniform in diameter from its root to the abdominal portion of the descending aorta. Furthermore, one may measure cardiac output using a thermodilution catheter or other techniques known to those of skill in the art. Finally, one may measure viscosity of the patient's blood by using known methods; for example, using a capillary viscosimeter. It is expected that in many cases, the application of this embodiment of the present method will provide a basis to more finely tune the system to more optimally operate the system to the patient's benefit. Other methods contemplated by the present invention may include steps to assess other patient parameters that enable a person of ordinary skill in the art to optimize the present system to ensure adequate mixing within the vascular system of the patient.

Alternative inventive methods that provide the benefits discussed herein include the steps of, prior to applying a shape change therapy, applying a blood supplementation system (such as one of the many examples described herein) to a patient, whereby the methods are designed to improve the ability to reduce the size and/or wall stress of the left ventricle, or both ventricles, thus reducing ventricular loading. Specifically, one example of such a method comprises the steps of providing a pump configured to pump blood at subcardiac rates, providing inflow and outflow conduits configured to fluidly communicate with non-primary blood vessels, fluidly coupling the inflow conduit to a non-primary blood vessel, fluidly coupling the outflow conduit to the same or different (primary or non-primary) blood vessel and operating the subcardiac pump in a manner, as described herein, to reduce the load on the heart, wherein the fluidly coupling steps may comprise anastomosis, percutaneous cannulation, positioning the distal end of one or both conduits within the desired terminal blood vessel or any combination thereof. The method further comprises, after sufficient reduction in ventricular loading, applying a shape change therapy in the form of, for example, a cardiac reshaping device, such as those referred to herein, or others serving the same or similar function, for the purpose of further reducing the size of and/or wall stress on one or more ventricles and, thus, the heart, and/or for the purpose of maintaining the patient's heart at a size sufficient to enhance recovery of the patient's heart.

Although the foregoing invention has been described in terms of certain preferred embodiments, other embodiments will be apparent to those of ordinary skill in the art. Additionally, other combinations, omissions, substitutions and modification will be apparent to the skilled artisan, in view of the disclosure herein. Accordingly, the present invention is not intended to be limited by the recitation of the preferred embodiments, but is instead to be defined by reference to the appended claims.

1. An intravascular extracardiac pumping system for increasing perfusion of tissue of a patient through a renal artery, the system comprising:
a pump configured to pump blood through the patient at subcardiac volumetric rates, said pump having an average flow rate that, during normal operation thereof, is substantially below that of the patient's heart when healthy, the pump configured to be positioned within the vasculature of a patient; and
a pump housing having:
an inflow portion defining an axis, the inflow portion fluidly coupled to the pump to direct blood to the pump, the inflow portion configured to be positioned within the vasculature of the patient; and
an outflow portion extending generally laterally from the axis of the inflow portion, the outflow portion fluidly coupled to the pump to direct blood away from the pump, the outflow portion configured to be at least partially positioned within the renal artery of the patient;
whereby the pump and the inflow and outflow portions are configured so as to be inserted subcutaneously into the vasculature in a minimally-invasive procedure.

2. The intravascular extracardiac pumping system of claim 1, wherein the inflow portion is configured to extend to a location midstream the renal artery and the heart of the patient when applied to the patient.

3. The intravascular extracardiac pumping system of claim 1, wherein the inflow portion is configured to extend to a vascular location proximate the aortic arch of the patient when applied to the patient.

4. The intravascular extracardiac pumping system of claim 1, wherein the inflow portion comprises an inlet end configured to extend to a location midstream the renal artery and a femoral artery when applied to the patient.

5. The intravascular extracardiac pumping system of claim 1, wherein the inflow portion comprises an inlet end configured to extend to a vascular location within an iliac artery when applied to the patient.

6. The intravascular extracardiac pumping system of claim 1, wherein the pump is configured to be positioned at a location midstream the heart of the patient and the renal artery.

7. The intravascular extracardiac pumping system of claim 6, wherein the pump is configured to direct blood in generally the same direction as the blood flowing adjacent the pump and outside the system.

8. The intravascular extracardiac pumping system of claim 1, wherein the pump is configured to be positioned at a location midstream the renal artery and a femoral artery of the patient.

9. The intravascular extracardiac pumping system of claim 8, wherein the pump is configured to direct blood generally counter to the direction of the blood flowing adjacent the pump and outside the system.

10. The intravascular extracardiac pumping system of claim 1, wherein the outflow portion comprises a first portion and a second portion, the first portion of the outflow portion configured to extend proximally from the pump to a location adjacent the renal artery and the second portion of the outflow portion configured to extend from the first portion of the outflow portion into the renal artery.

11. The intravascular extracardiac pumping system of claim 10, wherein the first portion of the outflow portion extends generally perpendicularly to the second portion of the outflow portion.

12. The intravascular extracardiac pumping system of claim 1, wherein the pump is a rotary pump.

13. The intravascular extracardiac pumping system of claim 1, wherein the pump is configured to operate in pulsatile fashion.

14. The intravascular extracardiac pumping system of claim 1, wherein the pump comprises an impeller.

15. The intravascular extracardiac pumping system of claim 14, wherein the impeller is helically shaped.

16. The intravascular extracardiac pumping system of claim 14, wherein the impeller is driven mechanically by a motor through a drive wire.

17. The intravascular extracardiac pumping system of claim 14, wherein the impeller is driven electromagnetically by a discrete electromagnetic drive.

18. The intravascular extracardiac pumping system of claim 17, wherein the electromagnetic drive is sized and configured to be implantable.

19. The intravascular extracardiac pumping system of claim 18, wherein the electromagnetic drive is sized and configured to be implantable within the patient's vasculature.

20. The intravascular extracardiac pumping system of claim 1, wherein the pump comprises a rotatable cable having means for directing blood axially along the cable.

21. The intravascular extracardiac pumping system of claim 1, wherein the pump comprises an Archemedes screw.

22. The intravascular extracardiac pumping system of claim 1, further comprising a pump driving means.

23. The intravascular extracardiac pumping system of claim 22, wherein the pump driving means is sized and configured to be implantable.

24. The intravascular extracardiac pumping system of claim 23, wherein the pump driving means is sized and configured to be implantable within the vasculature of a patient.

25. The intravascular extracardiac pumping system of claim 22, wherein the pump driving means comprises a drive wire.

26. The intravascular extracardiac pumping system of claim 22, wherein the pump driving means further comprises a motor.

27. The intravascular extracardiac pumping system of claim 22, wherein the pump driving means comprises an electromagnetic drive.

28. The intravascular extracardiac pumping system of claim 1, wherein the outflow portion comprises a first outflow portion and further comprising a second outflow portion, the first outflow portion being configured to be positioned within the renal artery and the second outflow portion being configured to be positioned within an artery of the patient.

29. The intravascular extracardiac pumping system of claim 28, wherein the second outflow portion is configured to be positioned within an iliac artery of the patient.

30. The intravascular extracardiac pumping system of claim 28, wherein the second outflow portion is configured to be positioned within a branch artery of the patient.

31. The intravascular extracardiac pumping system of claim 28, wherein the second outflow portion is configured to be positioned within the renal artery of the patient.

32. The intravascular extracardiac pumping system of claim 28, wherein the first outflow portion is configured to be positioned in a first renal artery and the second outflow portion is configured to be positioned in a second renal artery.

33. The intravascular extracardiac pumping system of claim 28, wherein the first outflow portion comprises a first portion and a second portion, the first portion of the first outflow portion configured to extend between the pump to a vascular location adjacent the renal artery and the second portion of the first outflow portion configured to extend from the first portion of the first outflow portion into the renal artery.

34. The intravascular extracardiac pumping system of claim 33, wherein the first portion of the first outflow portion extends generally perpendicularly to the second portion of the first outflow portion.

35. The intravascular extracardiac pumping system of claim 33, wherein the second outflow portion comprises a first portion and a second portion, the first portion of the second outflow portion configured to extend from the pump to a vascular location adjacent the artery and the second portion of the second outflow portion configured to extend from the first portion of the second outflow portion into the artery.

36. The intravascular extracardiac pumping system of claim 35, wherein the renal artery comprises a first renal artery and the artery comprises a branch artery.

37. The intravascular extracardiac pumping system of claim 35, wherein the renal artery comprises first renal artery and the artery comprises a second renal artery.

38. The intravascular extracardiac pumping system of claim 35, wherein the first portion of the second outflow portion extends generally perpendicularly to the second portion of the second outflow portion.

39. The intravascular extracardiac pumping system of claim 1, wherein the outflow portion comprises a first portion that extends from the pump to a first location adjacent the renal artery, a second portion that extends from the first portion into the renal artery, and a third portion that extends from the first portion into an artery.

40. The intravascular extracardiac pumping system of claim 39, wherein the third portion extends into an iliac artery.

41. The intravascular extracardiac pumping system of claim 39, wherein the third portion extends into a branch artery.

42. The intravascular extracardiac pumping system of claim 39, wherein the third portion extends into the renal artery.

43. The intravascular extracardiac pumping system of claim 39, wherein the first location is adjacent a first renal artery, the renal artery comprises a first renal artery, and the third portion extends into a second renal artery.

44. An intravascular extracardiac pumping system for increasing perfusion through a renal artery to tissues of a patient without any component thereof being connected to the patient's heart, the system comprising:
a means for pumping blood; and
a portion that houses the pumping means and is configured to direct blood from a location upstream of the pumping means to a first location within a first artery and a second location within a renal artery;
whereby the pumping means and the portion that houses the pumping means are configured to be insertable into a non-primary vessel subcutaneously in an minimally-invasive procedure for positioning within the patient's vasculature.

45. The intravascular extracardiac pumping system of claim 44, wherein the portion that houses the pumping means further comprises a first outflow portion and a second outflow portion, the first outflow portion being configured to be positioned within the first artery and the second outflow portion being configured to be positioned within the renal artery of the patient.

46. The intravascular extracardiac pumping system of claim 45, wherein the first outflow portion is configured to be positioned within an iliac artery of the patient.

47. The intravascular extracardiac pumping system of claim 45, wherein the first outflow portion is configured to be positioned within a branch artery of the patient.

48. The intravascular extracardiac pumping system of claim 45, wherein the first outflow portion is configured to be positioned within the renal artery of the patient.

49. The intravascular extracardiac pumping system of claim 45, wherein the first outflow portion is configured to be positioned in a first renal artery and the second outflow portion is configured to be positioned in a second renal artery.

50. The intravascular extracardiac pumping system of claim 45, wherein the second outflow portion comprises a first portion and a second portion, the first portion of the second outflow portion configured to extend between the pump to a vascular location adjacent the renal artery and the second portion of the second outflow portion configured to extend from the first portion of the second outflow portion into the renal artery.

51. The intravascular extracardiac pumping system of claim 50, wherein the first portion of the second outflow portion extends generally perpendicularly to the second portion of the second outflow portion.

52. The intravascular extracardiac pumping system of claim 50, wherein the first outflow portion comprises a first portion and a second portion, the first portion of the first outflow portion configured to extend from the pump to a vascular location adjacent the artery and the second portion of the first outflow portion configured to extend from the first portion of the first outflow portion into the artery.

53. The intravascular extracardiac pumping system of claim 52, wherein the renal artery comprises first renal artery and the first artery comprises a branch artery.

54. The intravascular extracardiac pumping system of claim 52, wherein the renal artery comprises a first renal artery and the artery comprises a second renal artery.

55. The intravascular extracardiac pumping system of claim 52, wherein the first portion of the first outflow portion extends generally perpendicularly to the second portion of the first outflow portion.

56. The intravascular extracardiac pumping system of claim 44, wherein the outflow portion comprises a first portion that extends from the pump to a third location adjacent the renal artery, a second portion that extends from the first portion to the first location within the renal artery, and a third portion that extends from the first portion to the second location in an artery.

57. The intravascular extracardiac pumping system of claim 56, wherein the third portion extends into an iliac artery.

58. The intravascular extracardiac pumping system of claim 56, wherein the third portion extends into a branch artery.

59. The intravascular extracardiac pumping system of claim 56, wherein the third portion extends into the renal artery.

60. The intravascular extracardiac pumping system of claim 56, wherein the third location is adjacent a first renal artery, the renal artery comprises a first renal artery, and the third portion extends to a second location in a second renal artery.

61. A method for treating a patient without connecting any component to the patient's heart, the method comprising the steps of:
inserting an inlet end of an inflow portion of an intravascular pumping system into the vasculature of a patient using a minimally invasive surgical procedure, the intravascular pumping system comprising a pump coupled with the inflow portion and an outflow portion coupled with the pump;
advancing the intravascular pumping system within the vasculature until the inlet end of the inflow portion is positioned at a first location within an artery and an outlet end of the outflow portion is positioned within a renal artery; and
operating said pump to pump blood through the renal artery to perfuse tissue at volumetric rates that are on average subcardiac.

62. The method of claim 61, wherein advancing the intravascular pumping system further comprises advancing the inlet end of the inflow portion to a location adjacent the aortic arch.

63. The method of claim 61, wherein advancing the intravascular pumping system further comprises advancing the inlet end of the inflow portion to a location adjacent the renal artery.

64. The method of claim 63, wherein advancing the intravascular pumping system further comprises advancing the inlet end of the inflow portion to a location midstream the renal artery and the heart.

65. The method of claim 64, wherein advancing the intravascular pumping system further comprises advancing the inlet end of the inflow portion to a location midstream the renal artery and a femoral artery.

66. The method of claim 61, wherein advancing the intravascular pumping system further comprises advancing the inlet end of the inflow portion to a location adjacent a femoral artery.

67. The method of claim 61, wherein advancing the intravascular pumping system further comprises advancing the inlet end of the inflow portion to a location within a femoral/iliac artery.

68. The method of claim 61, wherein advancing the intravascular pumping system further comprises positioning the outlet end of the outflow portion within a renal artery.

69. The method of claim 61, wherein the outflow portion comprises a first outflow portion, the outlet end comprises a first outlet end of the first outflow portion, and further comprising a second outflow portion having a second outlet end, and wherein advancing the intravascular pumping system further comprises positioning the first outlet end in the renal artery and positioning the second outlet end in an artery.

70. The method of claim 69, wherein advancing the intravascular pumping system further comprises positioning the first outlet end in the renal artery and positioning the second outlet end in an artery.

71. The method of claim 70, wherein advancing the intravascular pumping system further comprises positioning the second outlet end in a branch artery.

72. The method of claim 70, wherein advancing the intravascular pumping system further comprises positioning the second outlet end in the renal artery.

73. The method of claim 70, wherein advancing the intravascular pumping system further comprises positioning the first outlet end in a first renal artery and positioning the second outlet end in a second renal artery.

74. The method of claim 61, wherein the outflow portion comprises a first portion extending from the pump, a second portion extending from the first portion to a first outlet end, and a third portion extending from the first portion to a second outlet end.

75. The method of claim 74, wherein advancing the intravascular pumping system further comprises positioning the first outlet end in the renal artery and positioning the second outlet end in an artery.

76. The method of claim 75, wherein advancing the intravascular pumping system further comprises positioning the second outlet end in a branch artery.

77. The method of claim 75, wherein advancing the intravascular pumping system further comprises positioning the second outlet end in the renal artery.

78. The method of claim 75, wherein advancing the intravascular pumping system further comprises positioning the first outlet end in a first renal artery and positioning the second outlet end in a second renal artery.
